(19) United States
(12) Patent Application Publication
(10) Pub. No.: US 2026/0327578 A1
(43) Pub. Date: Oct. 22, 2026

(54) TRANSGLUTAMINASES AND USES THEREOF

(71) Applicant: CURIE CO. INC., Durham, NC (US)

(72) Inventors: Will SHINDEL, Durham, NC (US); Robert DICOSIMO, Doylestown, PA (US); Erika M. MILCZEK, Durham, NC (US); Ryan WOODYER, Dallas, GA (US); Marley ADAMEK, Cary, NC (US); Jaina Ashwinkumar BHAYANI, Raleigh, NC (US); John MCARTHUR, Morrisville, NC (US); Christian KASEY, Cary, NC (US)

(21) Appl. No.: 19/485,057

(22) PCT Filed: May 17, 2024
(86) PCT No.: PCT/US2024/030097

§ 371 Date: Nov. 17, 2025

Related U.S. Application Data
(60) Provisional application No. 63/503,288, filed on May 19, 2023.
(60) Provisional application No. 63/579,201, filed on Aug. 28, 2023.

(51) Int. Cl. C12N 9/10; A61K 8/66; A61Q 5/00; A61Q 5/06; C14C 3/26; C14C 11/00; D06M 16/00

(52) U.S. Cl. CPC C12N 9/1044; A61K 8/66; A61Q 5/004; A61Q 5/06; C12Y 203/02013; C14C 3/26; C14C 11/00; D06M 16/003; D06M 2200/12; D06M 2200/20; D06M 2200/35

(57) ABSTRACT

Transglutaminase variants with, inter alia, improved chemostability and/or productivity are disclosed herein. Uses of wild-type transglutaminase and transglutaminase variants are also disclosed herein.

### INCORPORATION BY REFERENCE

The sequence listing provided in the file named SL15939_0025_00304.xml with a size of 9,202 bytes, which was created on May 17, 2024, and which is filed herewith, is incorporated by reference in its entirety.

### FIELD OF INVENTION

The field pertains to transglutaminase variants comprising one or more amino acid substitutions that, inter alia, improve chemostability and/or productivity.

### BACKGROUND

Transglutaminases (Tgase, EC 2.3.2.13) are enzymes capable of catalyzing an acyl transfer reaction in which a γ-carboxy-amide group of a peptide bound glutamine residue is the acyl donor. Primary amino groups in a variety of compounds may function as acyl acceptors with the subsequent formation of monosubstituted γ-amides of peptide bound glutamine. When the ε-amino group of a lysine residue in a peptide chain serves as the acyl acceptor, the Tgases form intramolecular or intermolecular γ-glutamyl-ε-lysyl crosslinks. The catalytic reaction proceeds via glutamine deamination and formation of a protein-glutamyl-thioester at the active site of the enzyme. Nucleophilic attack by a lysyl ε-amino group of a second protein at the carbonyl moiety of the thioester intermediate generates isopeptide-crosslinked proteins that are largely resistant to proteolysis by common peptidases (Mariniello, et al. (2007) J Agr Food Chem.; 55:4717-4721). Bonds formed by a Tgase exhibit high resistance to proteolytic degradation (proteolysis).

Tgases can be found throughout all groups of organisms including prokaryotes, eukaryotes, and plants. Tgases in animals, for example, include blood coagulation factor XIII, which is a multi-domain protein and depends on calcium for regulation of enzyme function. Microbial transglutaminases, on the other hand, have only one single domain and do not depend on calcium for activity, i.e., Tgases of microbial origin are calcium-independent. Thus, microbial Tgases may provide major advantages for practical use.

Tgase has found many applications in biotechnology and in the food processing industry, where it has earned the moniker “meat glue.” The peptide crosslinking activity has shown useful for a variety of industrial purposes ranging from food processing, biotechnology, pharmaceuticals, medical devices, personal and household goods, and leather and textile treatment. The most commonly used Tgase is microbial transglutaminase from Streptomyces mobaraensis, having the amino acid sequence depicted in SEQ ID NO: 1.

Commercially available transglutaminase is produced by fermentation of Streptomyces mobaraensis. Transglutaminase is expressed as an inactive zymogen having a pro-peptide sequence at the N-terminus of the mature domain. The active enzyme is produced by removing the pro-peptide, i.e., the pro-domain, pro-sequence or pro-region, by proteolytic processing to afford the mature domain. Thus, the pro-peptide can be regarded as serving a regulatory function while the mature domain serves a catalytic function.

Due to its poor stability in solution, Tgase is typically formulated as a powder for commercial application and taken into solution or slurry at the time of use for crosslinking food protein. It was recently shown that in solution the enzyme may act on itself, resulting in the formation of Tgase-crosslinked aggregates, greatly reducing its enzymatic activity with exogenous proteins and peptides (Böhme et al. (2019) Amino Acids; 52 (2): 313-326). A need remains for transglutaminases with improved stability.

A solution stable Tgase also facilitates uses beyond food applications. Specifically, chemical crosslinkers such as glutaraldehyde, formaldehyde, chromium complexes, and triazine derivatives have been widely used to crosslink amino acid side chains for the creation of films, coatings, and natural fibers with improved durability (e.g. leather tanning, treatment of silk and wool). Additionally chemical crosslinkers have been used in hair treatments for straightening, style retention and color retention. However, these crosslinking chemicals have negative environmental and toxicity profiles. Tgase would provide a safer, greener alternative for creating protein-based coatings and films, tanning reagents for leather, and additionally as treatments for skin and hair. The formation of new isopeptide bonds to biologically relevant proteins on skin and hair such as collagen and keratin is a possible function for Tgase, as this enzyme is present in both the skin and hair follicles. However, wild-type Tgase only works poorly on these protein substrates under the manufacturing conditions required for textiles production, hair and skin care products, thereby creating the need for Tgase variants with improved activities and solution stability under the manufacturing conditions.

### SUMMARY

Herein we demonstrate that by engineering Tgase variants with fewer reactive glutamine and lysine residues, this self-deactivation activity can be overcome, providing chemostable variants of Tgase that are resistant to Tgase catalyzed crosslinking and deamidation activity. Furthermore, by reducing this self-deactivating pathway, hyperactive variants with improved productivity could be identified. The result is a family of solution stable Tgase variants with greater productivity to enable shipment of liquid formulations, addressing an unmet challenge in the field. In addition, we demonstrate herein that the disclosed Tgase variants, as well as wild-type Tgase, can be used in methods to form semipermanent protein films on objects such as hair, skin, textiles, or leather, such that the treated objects have improved properties such as color retention, reduced fraying, reduced wrinkling, and other benefits.

Transglutaminase variants are disclosed herein. In some embodiments, the transglutaminase variants have improved chemostability and/or productivity. Some of the variants demonstrate improvement in chemostability, rate, and/or productivity compared to the mature wild-type Tgase of Streptomyces mobaraensis (SEQ ID NO: 2).

In a first embodiment, the transglutaminase variant comprises the sequence of SEQ ID NO: 2 and further comprises at least one or more amino acid substitutions comprising one or more of the following: substitution of Q39 with N; substitution of Q40 with E, F, H, K, L, or N; substitution of K49 with H or R; substitution of Q50 with A, H, N, S or T; substitution of Q51 with A, C, D, E, G, I, K, P, or R; substitution of Q56 with A, E, or M; substitution of Q74 with A, D, E, F, G, H, I, L, M, N, R, S, T, V, or Y; substitution of K91 with A, C, E, H, M, N, or Y; substitution of K95 with I or R; substitution of Q124 with A, E, I, L, M, or N; substitution of K151 with R or Y; substitution of K152 with R; or substitution of Q328 with F, H, L, M, or P.

In a second embodiment, the transglutaminase variant comprises the sequence of SEQ ID NO: 2 comprising at least one or more amino acid substitutions comprising one or more of the following: substitution of A10 with T; substitution of K95 with E; substitution of S131 with E or I; substitution of V132 with L; substitution of Y146 with F; substitution of A166 with W; substitution of N282 with D; or substitution of H289 with I.

In a third embodiment, the transglutaminase variant of the first embodiment further comprises at least one or more amino acid substitutions comprising one or more of the following: substitution of A10 with C, Q or T; substitution of D14 with H, L, M, N, W, or Y; substitution of R15 with A, E, or T; substitution of D18 with E or T; substitution of G47 with H; substitution of R48 with M; substitution of K49 with E or T; substitution of Q74 with C; substitution of K95 with E; substitution of S131 with E or I; substitution of V132 with L; substitution of N134 with S or T; substitution of A136 with C or S; substitution of L137 with K or V; substitution of Y146 with F; substitution of L147 with E or M; substitution of E164 with F; substitution of P169 with E; substitution of F170 with I, L, or V; substitution of S199 with A or G; substitution of N282 with E, K, M, Q, or R; substitution of G283 with A; substitution of S284 with A, D, E, or P; substitution of H289 with E, I, L, Q, T, or V; substitution of H289 with I; or substitution of S299 with A, E, K, or V.

In a fourth embodiment, said transglutaminase variant of any of one of the first three embodiments comprises at least one or more amino acid substitutions comprising one or more of the following: substitution of Q39 with N; substitution of K49 with H or T; substitution of Q50 with A, H, S, or T; substitution of Q51 with A, E, I, or P; substitution of Q56 with E or M; substitution of Q74 with A, C, F, M, S, T, or V; substitution of K91 with E or H; substitution of K95 with E or I; substitution of Q124 with E, I, L, or N; substitution of K151 with R; or substitution of K152 with R.

In a fifth embodiment, the transglutaminase variant of any one of the first three embodiments comprises at least one or more amino acid substitutions comprising one or more of the following: substitution of Q50 with A, H, or S; substitution of Q51 with P; substitution of Q56 with E or M; substitution of Q74 with A, S, or V; substitution of K91 with E or H; substitution of Q124 with E, I, L, or N; substitution of K151 with R; or substitution of K152 with R.

In a sixth embodiment, the transglutaminase variant of the first embodiment comprises a combination of amino acid substitutions selected from: substitution of Q56 with M and Q74 with S; substitution of Q124 with E, I, or L, K151 with R, and K152 with R; substitution of Q50 with S, Q51 with E, and Q56 with E; and substitution of Q50 with S, Q74 with A, K91 with H, Q124 with L, K151 with R, and K152 with R.

In a seventh embodiment, the transglutaminase variant of the first embodiment comprises the following amino acid substitutions: substitution of Q50 with S, Q74 with A, K91 with H, Q124 with L, K151 with R, and K152 with R.

In an eighth embodiment, the transglutaminase variant of any one of the first, fourth, fifth, sixth, or seventh embodiments comprises at least variant one or more amino acid substitutions comprising one or more of the following: substitution of A10 with T; substitution of D14 with W; substitution of R15 with T; substitution of Q51 with E; substitution of A166 with W; substitution of F170 with I; substitution of N282 with M; substitution of G283 with A; substitution of H289 with I; and substitution of S299 with A or V.

In a ninth embodiment, the transglutaminase variant of the seventh embodiment further comprises a combination of amino acid substitutions selected from: substitution of N282 with M and G283 with A; substitution of D14 with W, A166 with W, N282 with M, G283 with A, and S299 with V; substitution of N282 with M, G283 with A, and S299 with V; substitution of D14 with W, N282 with M, G283 with A, and S299 with A; substitution of N282 with M, G283 with A, and S299 with V; substitution of A10 with T, N282 with M, G283 with A, and S299 with V; substitution of D14 with W, N282 with M, G283 with A, and S299 with V; substitution of R15 with T, N282 with M, G283 with A, and H289 with I; and substitution of Q51 with E, F170 with I, N282 with M, G283 with A, H289 with I, and S299 with V.

In a tenth embodiment, the transglutaminase variant from the first embodiment, comprises the following amino acid substitutions: substitution of Q50 with S, Q74 with A, K91 with H, Q124 with L, K151 with R, K152 with R, N282 with M, and G283 with A.

In an eleventh embodiment, the transglutaminase variant of the first embodiment comprises the following amino acid substitutions: substitution of D14 with W, Q50 with S, Q74 with A, K91 with H, Q124 with L, K151 with R, K152 with R, A166 with W, N282 with M, G283 with A, and S299 with V.

In a twelfth embodiment, the transglutaminase variant of the first embodiment comprises the sequence of SEQ ID NO: 3.

In a thirteenth embodiment, the transglutaminase variant of the first embodiment comprises the sequence of SEQ ID NO: 4.

In a fourteenth embodiment, the transglutaminase variant of the first embodiment comprises the sequence of SEQ ID NO: 5.

In a fifteenth embodiment, the transglutaminase variant of any one of the previous embodiments further comprises a substitution of S2 with an amino acid selected from C, D, E, F, H, I, K, L, M, N, P, Q, R, T, V, W, or Y.

In a sixteenth embodiment, the transglutaminase variant of the twelfth embodiment comprises one or more amino acid substitutions comprising one or more of the following substitutions: A10 with C, Q or T; D14 with H, L, M, N, W, or Y; R15 with A, E, or T; D18 with E or T; G47 with H; R48 with M; K49 with E or T; Q74 with C; K95 with E; S131 with E or I; V132 with L; N134 with S or T; A136 with C or S; L137 with K or V; Y146 with F; L147 with E or M; A166 with W; E164 with F; P169 with E; F170 with I, L, or V; S199 with A or G; N282 with E, K, M, Q, or R; G283 with A; S284 with A, D, E, or P; H289 with E, I, L, Q, T, or V; H289 with I; or substitution of S299 with A, E, K, or V; wherein the transglutaminase variant comprises no further amino acid substitutions or mutations with respect to SEQ ID NO: 3.

In a seventeenth embodiment, the transglutaminase variant of the thirteenth embodiment comprises one or more amino acid substitutions comprising one or more of the following substitutions: A10 with C, Q or T; D14 with H, L, M, N, W, or Y; R15 with A, E, or T; D18 with E or T; G47 with H; R48 with M; K49 with E or T; Q74 with C; K95 with E; S131 with E or I; V132 with L; N134 with S or T; A136 with C or S; L137 with K or V; Y146 with F; L147 with E or M; A166 with W; E164 with F; P169 with E; F170 with I, L, or V; S199 with A or G; N282 with E, K, Q, or R; S284 with A, D, E, or P; H289 with E, I, L, Q, T, or V; H289 with I; or S299 with A, E, K, or V, wherein the transglutaminase variant comprises no further amino acid substitutions or mutations with respect to SEQ ID NO: 4.

In an eighteenth embodiment, the transglutaminase variant of the seventeenth embodiment comprises 13 or less amino acid substitutions with respect to SEQ ID NO: 4.

In a nineteenth embodiment, the transglutaminase variant of the fourteenth embodiment comprises one or more amino acid substitutions comprising one or more of the following substitutions: A10 with C, Q or T; D14 with H, L, M, N, or Y; R15 with A, E, or T; D18 with E or T; G47 with H; R48 with M; K49 with E or T; Q74 with C; K95 with E; S131 with E or I; V132 with L; N134 with S or T; A136 with C or S; L137 with K or V; Y146 with F; L147 with E or M; E164 with F; P169 with E; F170 with I, L, or V; S199 with A or G; N282 with E, K, Q, or R; S284 with A, D, E, or P; H289 with E, I, L, Q, T, or V; H289 with I; and S299 with A, E, or K, wherein the transglutaminase variant comprises no further amino acid substitutions or mutations with respect to SEQ ID NO: 5.

In a twentieth embodiment, the mature transglutaminase sequence of the transglutaminase variant of any one of the first to nineteenth embodiments comprises an N-terminal methionine.

In a twenty-first embodiment, the transglutaminase variant of any one of the first to twentieth embodiments comprises a pro-sequence.

In a twenty-second embodiment, the transglutaminase variant comprises the sequence of SEQ ID NO: 6.

In a twenty-third embodiment, the composition comprises a transglutaminase variant according to any one of the preceding embodiments.

In a twenty-fourth embodiment, the composition of the twenty-third embodiment further comprises at least one antimicrobial enzyme, antimicrobial peptide, antimicrobial protein, and/or antimicrobial chemical.

In a twenty-fifth embodiment, the composition of the twenty-third embodiment further comprises a peptide, protein, or hydrolyzed protein at a concentration of about 0.04% to about 10%

In a twenty-sixth embodiment, the composition of the twenty-third embodiment further comprises a diamine molecule, wherein, optionally, the diamine molecule is one or more of 1,4-butanediamine, 1,5-pentanediamine, 1,6-hexanediamine, 1,8-octanediamine, and diethylenetriamine.

In a twenty-seventh embodiment, the method of preserving or increasing the shelf life of a product comprises incorporating one or more transglutaminase variants according to any one of the first to twenty-second embodiments and/or a composition according to the twenty-third embodiment or the twenty-fourth embodiment, into the product in an amount effective to provide antimicrobial activity in comparison to an identical product that does not comprise a transglutaminase variant.

In a twenty-eighth embodiment, the method of forming a semipermanent protein-film on an object comprises applying to the object a transglutaminase and/or the composition according to any one of the twenty-third, the twenty-fifth, and the twenty-sixth embodiments.

In a twenty-ninth embodiment, the object of the twenty-eighth embodiment is hair, skin, textile, or leather.

In a thirtieth embodiment, the composition of the twenty-eighth embodiment or the twenty-ninth embodiment comprises about 10 to about 10,000 ppm of the transglutaminase.

In a thirty-first embodiment, the method of retaining color and/or reducing color transfer on hair or skin comprises applying to the hair or the skin a composition comprising about 10 to about 10,000 ppm of a transglutaminase.

In a thirty-second embodiment, the method of reducing frizz and/or retaining style in hair comprises applying to the hair a composition comprising about 10 to about 10,000 ppm of a transglutaminase.

In a thirty-third embodiment, the method of improving hydrophobicity of hair comprises applying to the hair a composition comprising about 10 to about 10,000 ppm of a transglutaminase.

In a thirty-fourth embodiment, the method of retaining color and/or reducing color transfer on textile or leather comprises applying to the textile or the leather a composition comprising about 10 to about 10,000 ppm of a transglutaminase.

In a thirty-fifth embodiment, the method of strengthening and/or reducing fraying or peeling of textile comprises applying to the textile a composition comprising about 10 to about 10,000 ppm of a transglutaminase.

In a thirty-sixth embodiment, the method of improving water resistance and/or wrinkle resistance of textile comprises applying to the textile a composition comprising about 10 to about 10,000 ppm of a transglutaminase.

In a thirty-seventh embodiment, the method of any one of the thirty-first to thirty-sixth embodiments further comprises applying a peptide, protein, or hydrolyzed protein at a concentration of about 0.04% to about 10%.

In a thirty-eighth embodiment, the method of tanning hide comprises applying to the hide a transglutaminase variant at about 0.01% to about 5% w/w relative to the weight of the hide.

In a thirty-ninth embodiment, the method of the thirty-eighth embodiment further comprises applying to the hide a diamine molecule at about 0.1% to about 25% w/w relative to the weight of the hide, wherein, optionally, the diamine molecule is one or more of 1,4-butanediamine, 1,5-pentanediamine, 1,6-hexanediamine, 1,8-octanediamine, and diethylenetriamine.

In a fortieth embodiment, the method of repairing, coating, and/or improving water resistance of leather comprises applying to the leather a composition comprising about 10 to about 10,000 ppm of a transglutaminase.

In a forty-first embodiment, the transglutaminase of any one of the thirty-first to fortieth embodiments is a variant transglutaminase of any one of the first to twentieth embodiments.

### BRIEF DESCRIPTION OF THE SEQUENCES

The following sequences are disclosed in the XML-format Sequence Listing submitted herewith, which complies with World Intellectual Property Organization (WIPO) Standard ST.26 and 37 C.F.R. §§ 1.831-1.835. The symbols and format used for nucleotide and amino acid sequence data comply with the rules set forth in 37 C.F.R. § 1.832.

SEQ ID NO: 1 corresponds to the wild-type precursor zymogen form of Tgase (Pre-Pro-Tgase) from Streptomyces mobaraensis (Uniprot P84153), which includes the secretion peptide. The secretion peptide is denoted in bold (not underlined) text. The pro-peptide is denoted in bold, underlined text.

SEQ ID NO: 2 corresponds to the mature wild-type Tgase of Streptomyces mobaraensis.

SEQ ID NO: 3 corresponds to a variant of Streptomyces mobaraensis Tgase with improved chemostability.

SEQ ID NO: 4 corresponds to a variant of Streptomyces mobaraensis Tgase with improved chemostability and productivity.

SEQ ID NO: 5 corresponds to the variant of Streptomyces mobaraensis Tgase with improved chemostability and productivity.

SEQ ID NO: 6 corresponds to the recombinant zymogen-form Streptomyces mobaraensis Tgase (pro-Tgase) variant. The pro-Tgase variant includes an N-terminal methionine, and an additional methionine at the N-terminal of the mature sequence. The pro-peptide sequence is denoted in bold, underlined text.

SEQ ID NO: 7 corresponds to the variant pro-sequence of Streptomyces mobaraensis Tgase, which includes an N-terminal methionine.

### BRIEF DESCRIPTION OF THE FIGURES

FIG. 1 depicts the specific activity of a commercial Tgase compared to a variant Tgase.

### DETAILED DESCRIPTION

Transglutaminase (Tgase) enzymes that are mutant forms of the mature wild-type Tgase of Streptomyces mobaraensis (SEQ ID NO: 2) are disclosed herein. The Tgase variants of the mature wild-type Tgase of Streptomyces mobaraensis (SEQ ID NO: 2) comprise amino acid modifications that demonstrate improved chemostability and/or productivity relative to wild type Tgase (SEQ ID NO: 2).

All patents, patent applications, and publications cited herein are incorporated by reference in their entireties.

Words using the singular include the plural, and vice versa, unless the context clearly dictates otherwise.

In this disclosure, many terms and abbreviations are used. The following definitions apply unless specifically stated otherwise.

As used herein, the singular forms “a,” “an,” and “the” include plural references unless the context clearly dictates otherwise. For example, the term “a compound” or “at least one compound” may include a plurality of compounds, including mixtures thereof. The terms “a,” “an,” “the,” “one or more,” and “at least one,” for example, can be used interchangeably herein.

The term “about” as used herein can allow for a degree of variability in a value or range of at most within 10%, e.g., within 5%, or within 1% of a stated value or of a stated limit of a range.

The terms “and/or” and “or” are used interchangeably herein and refer to a specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B and/or C” is intended to encompass each of the following aspects: “A, B and C”; “A, B or C”; “A or C”; “A or B”; “B or C”; “A and C”; “A and B”; “B and C”; “A” (alone); “B” (alone); and “C” (alone).

The term “antimicrobial” refers to any agent or combination of agents that kills, inactivates, or inhibits the growth of any microbes such as bacteria, archaea, protozoa, fungi, algae, amoebas, viruses and the like. Antimicrobials can be biocides, biostats, disinfectants, boosters, and preservatives. In some case, antimicrobials can work in synergy or have additive effects which may enhance their effectiveness. Antimicrobials that demonstrate additive, potentiating, or synergistic effects are often referred to as boosters. Some antimicrobials are multifunctional agents, which may have multiple benefits including antimicrobial properties. Examples of antimicrobials include, but are not limited to, antimicrobial chemicals, antimicrobial polymers, antimicrobial proteins, antimicrobial enzymes, and antimicrobial peptides.

Applications where antimicrobials are uses include, but are not limited to, sterilization, disinfection, cleaning, and processing of surfaces and materials or extending a products shelf life.

The term “potentiate” refers to making effective or active or more effective or active.

The term “broad spectrum antimicrobial” is one that acts against a wide range of microorganisms, for example Gram-positive bacteria, Gram-negative bacteria, yeast, mold, viruses, etc.

It should be noted that the broad spectrum antimicrobial and/or preservative compositions disclosed herein may be used in a variety of applications such as personal care, household, industrial, institutional, oil and gas, marine, food and beverage, agricultural, animal, and human nutrition, water purification and the like.

The terms “microorganism” and “microbe” are used interchangeably herein and refer to any living thing that is so small that it can be seen with a microscope, i.e., a microscopic organism. Microbes may exist in a single-celled form or in a colony of cells or in a biofilm. Microbes include eukaryotes and prokaryotes such as bacteria, archaea, protozoa, fungi, algae, amoebas, viruses and the like.

The term “preservative” refers to a substance or agent that is added to a product to prevent decomposition or contamination by microbial growth or by undesirable chemical changes. Also included in “preservatives” are antioxidants and oxygen removal substances. Examples of such antioxidants and oxygen removal substances include, but are not limited to, ascorbic acid, superoxide dismutase, catalase and the like. Examples of products to which preservatives may be added include, but are not limited to, food products, beverages, pharmaceutical drugs, paints, biological samples, cosmetics, wood, household cleaning products, personal care products and the like.

The term “shelf life” refers to the length of time for which an item (e.g., a product as described herein) remains usable, saleable, or fit for consumption.

Preservatives are antimicrobial ingredients added to product formulations to maintain the microbiological safety of the products by inhibiting the growth of and reducing the amount of microbial contaminants. US Pharmacopeia (USP) and the Personal Care Products Counsel (PCPC) have published protocols for acceptable microbial survival for preservatives in cosmetics and personal care products, such as USP <51> and USP <61> (Antimicrobial Effectiveness Test) as well as PCPC M-3 (also available are PCPC M-1, PCPC M-2, PCPC M-4, PCPC M-5, PCPC M-6, and PCPC M-7).

The effectiveness of the antimicrobial systems disclosed herein may also be determined by MIC (minimum inhibitory concentration) against a variety of microbes. MIC is defined as the lowest concentration of an antimicrobial that will inhibit the growth of a microorganism.

Checkerboard analysis is used to determine the impact on potency of the combination of antimicrobials in comparison to their individual activities. This comparison is often represented as the Fractional Inhibitory Concentration (FIC) index value. The FIC index value takes into account the combination of antimicrobials that produces the greatest change from the individual antimicrobial's MIC. In the checkerboard assay, rows and columns represent 2-fold serial dilutions of antimicrobial-1 and antimicrobial-2. Two compounds are tested in the serial dilutions, and the concentration of each compound is tested individually and in combination. Additionally, this method can be applied not only in combination with antimicrobial compounds, but also in combination with compounds that typically do not have antimicrobial properties.

The term “decontamination” used herein describes a process wherein a raw material, final product, or a waste stream (produced during manufacturing) is treated with an antimicrobial to reduce microbial contamination or bioburden (i.e., the number of living microorganisms present). Success of decontamination of a product can be measured by USP <61>, which involves quantitative testing for enumeration of total bacteria, yeast, or mold present.

An “aseptilase” is an enzyme that can prevent or reduce the risk of microbial contamination through antimicrobial action. In other words, aseptilases act on one or more class(es) of microorganisms, for example Gram-positive and -negative bacteria, fungi, and/or viruses. Aseptilase activity may be derived from the enzyme acting directly on the cell or a cellular component, producing an antimicrobial compound or precursor as a product of catalysis, consuming one or more essential nutrients through its activity, potentiating other antimicrobials present in the environment, or any combination thereof. It has been shown that crosslinking enzymes, such as transglutaminase, can be evolved for aseptilase activity (WO 2020/181099A1, WO 2021/183680A1, WO 2021/231705A1, and WO 2022/055902). Using directed evolution, naturally occurring enzyme activity can be evolved to have novel and/or enhanced aseptilase activity.

The term “semipermanent” or “permanent” as used herein refers to the length of duration of a beneficial property conferred to an object treated with disclosed Tgase compositions. For example, hair treated with a disclosed Tgase may have improved color retention or decreased color transfer for a longer, or semipermanent, duration of time than untreated hair. In some cases “semipermanent” or “permanent” refers to the durability of a protein film formed by treating an object with a Tgase, compared to an object not treated with a Tgase composition. In some cases “semipermanent” or “permanent” refers to the film or coating lasting through multiple washes with a cleanser or surfactant.

The term “hydrolyzed protein” refers to a protein that has been hydrolyzed enzymatically or through acid-base hydrolysis. Non-limiting examples of hydrolyzed proteins include hydrolyzed keratin, collagen, gelatin, sodium caseinate, casein, silk protein, potassium cocoyl hydrolyzed collagen, or plant-based proteins (examples include rice protein, barley protein, baobab protein, quinoa protein, mung bean protein, pea protein, and soy protein). Additionally, a hydrolyzed protein could be combination of amino acids and peptides from a variety of sources including fermentation (such as arginine HCl, serine, threonine).

The term “semipermanent protein-film” refers to a proteinaceous substance or agent that is applied to the surface of a product, skin, or hair to repair damage or to change, enhance, or improve the aesthetic or functional properties. Examples of damage include, but are not limited to, decoloration, staining or soiling, abrasion or tearing, and water damage. Examples of a proteinaceous substance include, but are not limited to, casein, or gelatin, or collagen, or keratin-based coatings. Furthermore, these coatings may be enhanced by binders, including non-limiting examples such as Aziridines, formaldehyde, and enzymes. Examples of products to which protein-film coatings may be applied include, but are not limited to, textiles-such as wool and cotton, natural materials-such as leather, and synthetic fibers-such as polyester. Non-limiting examples of protein-film applications include automobile upholstery, clothing, and paints. Semipermanent protein-films could be made by incorporating film formers and Tgase to improve longevity of the film former.

“Film formers” or “film-forming agents” are a group of chemicals or polymers that leave a pliable, cohesive, and continuous covering over the hair or skin when applied to their surface. Inclusion of a film former in a cosmetic composition can improve various properties, such as, for example, shine, adhesion, and long wear. These films could have hydrophilic properties. The films could have style retention properties. The films could have protective properties. Non-limiting examples of film-forming agents include proteins, hydrolyzed proteins, polyquaternium compounds, silicones, polyvinylpyrrolidone, acrylates, acrylamides, and copolymers. Film formers are commonly found as ingredients of cosmetics, particular hair-care products, but also moisturizers and other skin-care products.

“Textiles” are materials made from fibers, thin threads or filaments that are natural or synthetic or a combination of both. Textile fibers can be classified in natural (organic) fibers and man-made (synthetic, industrial) fibers, there is an enormous variety of textile fiber types available. Textile fibers can be spun into yarn and are processed into fabric by different methods such as weaving, knitting, felting etc. Herein, ideal embodiments of textiles are in the form of wool, silk, cashmere, other protein-based fibers, and nylon.

“Textile coatings” usually provide material layers which adhere to the textile structures. Textile coatings are generally used to change, enhance, or improve the aesthetic or functional properties of the textile. A typical textile coating formulation generally contains polymeric binder(s) along with other additives (such as colorants, adhesion promoter, biocide, plasticizers, etc.) Rubber, plastic, and vinyl coatings are just a few examples. Fabric coatings are capable of enhanced properties such as hydrophobicity, antimicrobial resistance, Ultraviolet protection, and elasticity. Herein, textile coatings are created from crosslinking peptide, protein, or hydrolyzed proteins to the surface of a textile or leather, optionally, with diamine molecules.

The term “amino acid” refers to the basic chemical structural unit of a protein, peptide, or polypeptide. The following abbreviations used herein to identify specific amino acids can be found in Table 1.

TABLE 1
One- and Three-Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or | Xaa | X |
| as defined herein | | |

The term “chemostability” disclosed herein refers to the ability of the enzyme to maintain its activity over time e.g., due to reduced self-deactivation activity. Chemostability may be assessed as described in Example 3.

The terms “comprises,” “comprising,” “includes,” “including,” “having” and their conjugates are used interchangeably and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “derived from” encompasses the terms “originated from,” “obtained from,” “obtainable from,” “isolated from,” “purified from,” and “created from,” and generally indicates that one specified material finds its origin in another specified material or has features that can be described with reference to another specified material.

The terms “isolated,” “purified,” “separated,” and “recovered” as used herein refer to a material (e.g., a protein, nucleic acid, or cell) that is removed from at least one component with which it is naturally associated. For example, these terms may refer to a material which is substantially or essentially free from components which normally accompany it as found in its native state, such as, for example, an intact biological system. An isolated nucleic acid molecule includes a nucleic acid molecule contained in cells that ordinarily express the nucleic acid molecule, but the nucleic acid molecule is present extra-chromosomally or at a chromosomal location that is different from its natural chromosomal location.

Throughout this application, various embodiments can be presented in a range format. It should be understood that the description in range format is merely for convenience and brevity and should not be construed as an inflexible limitation on the scope of the embodiments described herein. Accordingly, the description of a range should be considered to have specifically disclosed all the possible subranges as well as individual numerical values within that range. For example, description of a range, such as from 1 to 6 should be considered to have subranges such as from 1 to 2, from 1 to 3, from 1 to 4 and from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5 and 6. This applies regardless of the breadth of the range.

The terms “mature,” “active,” and “activated” are used interchangeably herein. A mature form of an enzyme, protein, polypeptide, or peptide refers to the functional form of the protein, polypeptide, or enzyme without a signal, silencing, or chaperoning pro-peptide sequence. Additionally, the mature enzyme may be truncated relative to the mature sequence while maintaining the desired activity (e.g., Tgase having or capable of reacting with amino acids, peptides and/or proteins). Additionally, the mature enzyme may have additional amino acids at either the N-terminus or C-terminus that do not adversely affect activity. Examples of such amino acids include but are not limited to affinity tags, fragments of an adjacent domain such as a pro-peptide or signal peptide, a linker region, a start codon, or a combination thereof.

The term “mutation” herein refers to a change introduced into a parental sequence, including, but not limited to, modifications such as residue substitutions, insertions, or deletions (including truncations), thereby producing a “variant.” The consequences of a mutation include, but are not limited to, the creation of a new character, property, function, phenotype, or trait not found in the protein encoded by the parental sequence.

“Optional” or “optionally” means that the subsequently described event, circumstance, or material may or may not occur or be present, and that the description includes instances where the event, circumstance, or material occurs or is present and instances where it does not occur or is not present.

The terms “percentage of sequence identity,” “percent identity,” “percent identical,” and “percent sequence identity” refer to comparisons between polynucleotide sequences or polypeptide sequences, and are determined by comparing two optimally aligned sequences over a comparison window, wherein the portion of the polynucleotide or polypeptide sequence in the comparison window may comprise mismatches, additions, and/or deletions (i.e., gaps) as compared to the reference sequence for optimal alignment of the two sequences. The percentage is calculated by determining the number of positions at which either the identical nucleic acid base or amino acid residue occurs in both sequences, or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (See e.g., Altschul et al., (1990) J. Mol. Biol.; 215:403-410; and Altschul et al., (1977) Nucl. Acids Res.; 3389-3402). Software for performing BLAST analyses is publicly available through the National Center for Biotechnology Information website.

One of ordinary skill in the art will appreciate that alterations in a gene at a given site which result in the production of a chemically equivalent amino acid, but do not affect the functional properties of the encoded protein, are common.

Enzymatic “productivity” is a measure of product formation or substrate disappearance over time, at a prescribed temperature under specified reaction conditions. It is the measure both the durability and reaction yield of the enzyme variant. In this instance, productivity is defined as total product formed over 24-hours at a constant substrate concentration when the enzyme is the rate limiting factor. The time is defined as 24 hours, which represents approaching or surpassing burnout, denaturation, or self-reaction of the enzyme variant. In other words, variants with higher productivity deliver a higher product yield in 24-hours than variants with lower productivity produce in the same period of time. “Rate” refers to the initial rate for an enzymatic reaction, at a prescribed temperature under specified reaction conditions. The enzyme produces product at an initial rate that is approximately linear for a short period after the start of the reaction. When concentrations of enzyme and substrate are the same across the sample set, initial rates between enzyme variants can be compared. In this instance, the linear range for production of product occurs in one hour. Enzyme variants with faster initial rates deliver a higher product yield in one hour than variants with slower initial rates produce at the same time-period. In some cases, improvements to initial rates also deliver a more productive variant. In some cases, improvements to initial rates result in faster decay of the enzyme variant reducing overall enzymatic productivity. Productivity and rate may be assessed as described in Example 2.

The terms “pro-peptide,” “pro-domain,” and “pro-region” are used interchangeably herein and refer to a N-terminal peptide leader sequence that is cleaved to afford active transglutaminase from the native zymogen form. A pro-peptide may contain more than one cleavage site and partial cleavage of the pro-peptide may also afford active transglutaminase. Alternatively, this pro-peptide or a fragment thereof can be added exogenously or co-expressed as a discrete polypeptide independent of the mature transglutaminase. The pro-peptide can be regarded as serving a regulatory function while the mature domain serves a catalytic function. Pro-peptides generally are recognized to have four major functions: 1) pro-peptides can function as intramolecular chaperones or folding assistants by determining the three-dimensional structure of a protein; 2) pro-peptides can function as inhibitors or activation peptides; 3) pro-peptides can direct protein sorting into specific cellular compartments or extra-cellular space and 4) pro-peptides can mediate the precursor interaction with other molecules (such as peptides, proteins, and polysaccharides) or supramolecular structures (e.g., cell walls). A single pro-peptide can perform several or even all these functions. The pro-peptide may be the naturally-occurring pro-peptide sequence for the mature Tgase, or it may be engineered, e.g. to enhance pH-responsive, binding properties, productivity, and/or thermostability, among other qualities, as disclosed in U.S. 63/344,392. In some embodiments, the pro-peptide is the naturally occurring pro-sequence from the zymogen form of the Streptomyces mobaraensis Tgase (shown in bold, underlined text of SEQ ID NO: 1). In some embodiments, the pro-peptide comprises an N-terminal methionine. In some embodiments, the pro-peptide has the sequence of SEQ ID NO: 7.

A “protease” (also called a peptidase or proteinase) refers to enzymes capable of cleaving peptide bonds. Proteases are any of various enzymes, such as endopeptidases and exopeptidases, that catalyze the hydrolytic breakdown of proteins into peptides and amino acids. Proteases can be classified into seven broad groups: serine proteases, cysteine proteases, threonine proteases, aspartic proteases, glutamic proteases, metalloproteases, and asparagine peptide lyases. Proteases can be found in animals, plants, bacteria, fungi, archaea, and viruses. The terms “protease,” “peptidase,” and “proteinase” are used interchangeably herein.

The term “reversible inhibitor” refers to an inhibitor that inactivates an enzyme through noncovalent, more easily reversed, interactions. Unlike an irreversible inhibitor, a reversible inhibitor can dissociate from the enzyme. Reversible inhibitors include competitive inhibitors, noncompetitive inhibitors, and uncompetitive inhibitors.

The terms “signal sequence” and “signal peptide” are used interchangeably herein and refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported. They can be called pre-pro-peptides or pre-pro-proteins.

The term “transglutaminase” (Tgase, EC2.3.2.13) refers to a family of enzymes that catalyze the formation of an isopeptide bond between a primary amine, for example, the s-amine of a lysine molecule, and the acyl group of a protein- or peptide-bound glutamine. Transglutaminases may catalyze a transamidation reaction between glutamyl and lysyl side chains of target proteins. Proteins possessing Tgase activity have been found in microorganisms, plants, and animals. Tgases are widely distributed in various organs, tissues, and bodily fluids. Tgases also form extensively crosslinked, generally insoluble, protein biopolymers that are needed for an organism to create barriers and stable structures. As used herein, a “mature Tgase” is a Tgase having or capable of reacting with amino acids, peptides and/or proteins.

Tgases of microbial origin, unlike eukaryotic Tgases, are calcium-independent, which represents a major advantage for their practical use. Microbial transglutaminase (EC 2.3.2.13) is one of the most extensively studied industrial enzymes for protein functionalization and protein crosslinking because of its ability to polymerize or functionalize proteins through the formation of a stable ε-(γ-glutamyl) lysine isopeptide bond without the constraint of a consensus sequence or additional cofactors.

Related (and derivative) proteins encompass “variant,” “mutant,” or “modified” proteins, which terms are used interchangeably herein. Variant (i.e., mutant or modified) proteins differ from another (i.e., parental) protein or from one another due to modifications in one or more amino acid residues but retain at least a degree of one functional property of a parent molecule. For example, a variant may include one or more amino acid modifications such as one or more amino acid deletions/truncations, insertions, or substitutions as compared to the parental protein from which it is derived.

Alternatively or additionally, variants may have a specified degree (percentage) of sequence identity with a reference protein or nucleic acid using the BLAST percent identity algorithms. For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid or nucleic acid, respectively, sequence identity with a reference sequence and integer percentage therebetween. In some embodiments, the variant Tgase has an amino acid sequence having at least 85%, 86%, 87%, 88%, 89%, 90%, 91%, 92%, 93%, 94%, 95%, 96%, 97%, 98%, or 99% amino acid sequence identity with a reference sequence. In some embodiments, the reference Tgase sequence is SEQ ID NO: 2. In some embodiments, the reference Tgase sequence is SEQ ID NO: 3, SEQ ID NO: 4, or SEQ ID NO: 5.

Variant Tgases disclosed herein may have an amino acid sequence that differs from a reference sequence by a certain number of amino acids. In some embodiments, a variant Tgase may comprise 20, 19, 18, 17, 16, 15, 14, 13, 12, 11, 10, 9, 8, 7, 6, 5, 4, 3, 2, or 1 amino acid substitutions compared to a reference Tgase sequence. In some embodiments, the reference Tgase sequence is SEQ ID NO: 2. In some embodiments, the reference Tgase sequence is SEQ ID NO: 3, SEQ ID NO: 4, or SEQ ID NO: 5.

The term “wild-type” in reference to an amino acid sequence or nucleic acid sequence indicates that the amino acid sequence or nucleic acid sequence is a native or naturally-occurring sequence (e.g., SEQ ID NO: 2). As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant/engineered nucleic acids and protein sequences produced in the laboratory or modification of the wild-type sequence).

The terms “zymogen” and “proenzyme” are used interchangeably herein and refer to an inactive precursor of an enzyme, which may be converted into an active or mature enzyme by post-translational modification, for example, by catalytic action, such as via proteolytic cleavage of a pro-peptide sequence.

### Transglutaminase Variants

The Tgase variants disclosed in Tables 2-6 are variants of the wild-type mature Tgase amino acid sequence set forth in SEQ ID NO: 2. As noted above, mutations can be named by the one letter code for the parent amino acid, followed by a position number and then the one letter code for the variant amino acid. For example, mutating glycine (G) at position 87 to serine(S) is represented as “G087S” or “G87S.” The position number is determined by the native N-terminus of the wild-type mature Tgase sequence set forth in SEQ ID NO: 2; however, the variants described herein may comprise additional N-terminal residues, such as residues derived from their pro-peptides.

The variants in Table 2 have similar or improved activity compared to the wild type Tgase of SEQ ID NO: 2. Table 2 variants include substitutions of lysine (K) and glutamine (Q) residues in the wild type Tgase sequence.

TABLE 2
Variants containing mutations of glutamine and
lysine residues relative to SEQ ID NO: 2

| Position | Variant |
|---|---|
| Q39 | Q39N |
| Q40 | Q40(E, F, H, K, L, N) |
| K49 | K49(R, T) |
| Q50 | Q50(A, H, N, S, T) |
| Q51 | Q51(A, C, D, E, G, I, K, P, R) |
| Q56 | Q56(A, E, M) |
| Q74 | Q74(A, C, D, E, F, G, H, I, L, M, N, R, S, T, V, Y) |
| K91 | K91(A, C, E, H, M, N, Y) |
| K95 | K95(I, R) |
| Q124 | Q124(A, E, I, L, M, N) |
| K151 | K151(R, Y) |
| K152 | K152R |
| Q328 | Q328(F, H, L, M, P) |

In some embodiments, the Tgase variant comprises one or more substitutions depicted in Table 2.

The Tgase variants disclosed in Table 3 are substitution variants of the wild-type mature Tgase amino acid sequence set forth in SEQ ID NO: 2 that have increased activity relative to the wild type mature Tgase of SEQ ID NO: 2. The substitutions in Table 3 can be used in combination with the activity substitutions in Table 2, which are believed to aid in chemostability.

TABLE 3
Substitution variants directed to improved
activity relative to SEQ ID NO: 2

| Position | Variant |
|---|---|
| A10 | A10T |
| K95 | K95E |
| S131 | S131(E, I) |
| V132 | V132L |
| Y146 | Y146F |
| A166 | A166W |
| N282 | N282D |
| H289 | H289I |

In some embodiments, the Tagase variant comprises one or more substitutions depicted in Table 3. In some embodiments, the Tgase variant comprises one or more substitutions depicted in Table 3 in combination with one or more substitutions depicted in Table 2.

Tgase variants known in the art may also be used in combination with the activity/chemostability substitutions in Table 2 and/or activity in Table 3. The Tgase variants disclosed in Table 4 are substitution variants of the wild-type mature Tgase amino acid sequence set forth in SEQ ID NO: 2 for enhanced activity and are disclosed in WO 2021/183680 and WO 2021/231705, which are incorporated by reference herein. In some embodiments, the substitutions in Table 4 can be used in combination with the activity/chemostability substitutions in Table 2 and/or activity substitutions in Table 3. As a person of skill in the art would appreciate, multiple substitutions at the same position cannot be used in combination (e.g., A10T cannot be used in combination with A10C).

TABLE 4
Substitution variants directed to improved
activity relative to SEQ ID NO: 2

| Position | Variant |
|---|---|
| A10 | 10(C, Q) |
| D14 | D14(H, L, M, N, W, Y) |
| R15 | R15(A, E, T) |
| D18 | D18(E, T) |
| G47 | G47H |
| R48 | R48M |
| K49 | K49(E, T) |
| Q74 | Q74C |
| N134 | N134(S, T) |
| A136 | A136(C, S) |
| L137 | L137(K, V) |
| L147 | L147(E, M) |
| E164 | E164F |
| P169 | P169E |
| F170 | F170(I, L, V) |
| S199 | S199(A, G) |
| N282 | N282(E, K, M, Q, R) |
| G283 | G283A |
| S284 | S284(A, D, E, P) |
| H289 | H289(E, I, L, Q, T, V) |
| S299 | S299(A, E, K, V) |

The Tgase variants disclosed in Table 5 are substitution variants of the wild-type mature Tgase amino acid sequence set forth in SEQ ID NO: 2 that have enhanced stability compared to wild-type Tgase.

TABLE 5
Stability variants of Tgase relative to SEQ ID NO: 2

| Variant | | |
|---|---|---|
| Name | Sequence (Relative to SEQ ID NO. 2) | Stability |
| S1 | S2P Q50H | ++ |
| S2 | S2P Q74V | ++ |
| S3 | S2P Q50A | ++ |
| S4 | S2P Q51P | + |
| S5 | S2P Q39N Q51E | + |
| S6 | S2P Q56M Q74S | ++ |
| S7 | S2P K49T Q74M | + |
| S8 | S2P K49T Q74F | + |
| S9 | S2P Q51I K95I | + |
| S10 | S2P Q124E K151R K152R | ++ |
| S11 | S2P Q124I K151R K152R | ++ |
| S12 | S2P Q124L K151R K152R | ++ |
| S13 | S2P Q124N K151R K152R | + |
| S14 | S2P Q50S Q51E Q56E | ++ |
| S15 | S2P Q51P Q56M Q74A K91E | ++ |
| S16 | S2P Q50S Q74A K91H Q124L K151R K152R | ++ |
| S17 | S2P Q50S Q74A K91H Q124N K151R K152R | + |
| S18 | S2P K49H Q50A Q74C Q124L K151R K152R | + |
| S19 | S2P K49H Q50A Q74C Q124N K151R K152R | + |
| S20 | S2P Q51A Q74T K95I Q124L K151R K152R | + |
| S21 | S2P Q50T Q51A Q74M K95E Q124L K151R K152R | + |

Table 5 depicts variants of Streptomyces mobaraensis Tgase and improvement to stability relative to wild-type mature Tgase (SEQ ID NO. 2) as determined in Example 3. Numbering of amino acid positions is in reference to the mature S. mobaraensis Tgase sequence depicted in SEQ ID NO. 2. A “+” indicates an improvement between 1.2 and 2-fold. A “++” indicates an improvement greater than 2-fold.

In some embodiments, the Tgase variant comprises a combination of substitutions depicted in Table 5, further comprising one or more substitutions depicted in Table 3 and/or one or more substitutions depicted in Table 4. In some embodiments, the Tgase variant comprises a combination of substitutions depicted in Table 5, further comprising one or more substitutions depicted in Table 2, Table 3, and/or Table 4. As a person of skill in the art would appreciate, multiple substitutions at the same position cannot be used in combination (e.g., A10T cannot be used in combination with A10C).

In some embodiments, the Tgase variant has greater than 2-fold stability relative to wild-type mature Tgase (SEQ ID NO: 2). In some embodiments, the Tgase variant has greater than 1.2-fold, 1.3-fold, 1.4-fold, 1.5-fold, 1.6-fold, 1.7-fold, 1.8-fold, or 1.9-fold stability relative to wild-type mature Tgase. In some embodiments, the improved stability of the Tgase variants is due to greater chemostability relative to wild-type mature Tgase.

In some embodiments, the Tgase variant comprises one or more amino acid substitutions comprising one or more of the following substitutions with respect to SEQ ID NO: 2: substitution of Q50 with A, H, or S; substitution of Q51 with P; substitution of Q56 with E or M; substitution of Q74 with A, S, or V; substitution of K91 with E or H; substitution of Q124 with E, I, L, or N; substitution of K151 with R; or substitution of K152 with R. In some embodiments, the Tgase variant comprises a combination of amino acid substitutions with respect to SEQ ID NO: 2 selected from: substitution of Q56 with M and Q74 with S; substitution of Q124 with E, I, or L, K151 with R, and K152 with R; substitution of Q50 with S, Q51 with E, and Q56 with E; and substitution of Q50 with S, Q74 with A, K91 with H, Q124 with L, K151 with R, and K152 with R. In some embodiments, the Tgase variant comprises the following combination of amino acid substitutions with respect to SEQ ID NO: 2: substitution of Q50 with S, Q74 with A, K91 with H, Q124 with L, K151 with R, and K152 with R. In some embodiments, the Tgase variant comprises the combination of substitutions in variant S16 in Table 5. In some embodiments, the Tgase variant comprises a substitution of S2 with P. In some embodiments, the Tgase variant comprises an N-terminal methionine. In some embodiments, the Tgase variant comprises a C-terminal polyhistidine sequence. In some embodiments, the Tgase variant comprises the sequence of SEQ ID NO: 3.

The Tgase variants disclosed in Table 6 are substitution variants of the wild-type mature Tgase sequence set forth in SEQ ID NO: 2 that have enhanced productivity and/or rate relative to the wild-type mature Tgase of SEQ ID NO: 2. The Tgase variants disclosed in Table 6 include the chemostability substitutions present in variant S16 of Table 5.

TABLE 6
Substitutions related to productivity and rate
of Tgase variants relative to SEQ ID NO: 2

| Variant | | | |
|---|---|---|---|
| Name | Sequence (Relative to SEQ ID NO. 2) | Productivity | Rate |
| A1 | S2P Q50S Q74A K91H Q124L K151R K152R N282R | + | − |
| A2 | S2P Q50S Q74A K91H Q124L K151R K152R N282E | − | + |
| | S284A | | |
| A3 | S2P Q50S Q74A K91H Q124L K151R K152R N282M | + | − |
| | G283A | | |
| A4 | S2P Q50S Q74A K91H Q124L K151R K152R N282Q | + | − |
| | S284A H289I | | |
| A5 | S2P Q50S Q74A K91H Q124L K151R K152R N282D | + | − |
| | S284A H289I S299E | | |
| A6 | S2P D14W Q50S Q74A K91H Q124L K151R K152R | ++ | +++ |
| | A166W N282M G283A S299V | | |
| A7 | S2P Q50S Q74A K91H Q124L K151R K152R N282M | + | + |
| | G283A S299V | | |
| A8 | S2P D14W Q50S Q74A K91H Q124L K151R K152R | + | ++ |
| | N282M G283A S299A | | |
| A9 | S2P Q50S Q74A K91H K95E Q124L K151R K152R | + | + |
| | N282M G283A S299V | | |
| A10 | S2P A10T Q50S Q74A K91H Q124L S131I K151R | + | + |
| | K152R N282M G283A S299V | | |
| A11 | S2P D14W Q50S Q74A K91H Q124L S131I K151R | + | + |
| | K152R N282M G283A S299V | | |
| A12 | S2P R15T Q50S Q74A K91H Q124L K151R K152R | + | + |
| | N282M G283A H289I | | |
| A13 | S2P Q50S Q51E Q74A K91H Q124L K151R K152R | + | + |
| | F170I N282M G283A H289I S299V | | |
| A14 | S2P Q50S Q51E Q74A K91H Q124L Y146F K151R | + | − |
| | K152R N282M G283A H289I S299V | | |
| A15 | S2P Q50S Q74A K91H K95E Q124L K151R K152R | − | + |
| | N282M G283A | | |
| A16 | S2P D18T Q50S Q74A K91H Q124L S131R V132L | − | ++ |
| | K151R K152R N282M G283A | | |
| A17 | S2P D14N Q50S Q74A K91H Q124L K151R K152R | − | + |
| | N282M G283A | | |
| A18 | S2P Q50S Q51A Q74A K91H Q124L K151R K152R | − | + |
| | N282M G283A H289I | | |

Table 6 depicts variants of Streptomyces mobaraensis Tgase and improvement to productivity and/or rate relative to wild-type Tgase (SEQ ID NO. 2) as determined in Example 3. Numbering of amino acid positions is in reference to the mature S. mobaraensis Tgase sequence depicted in SEQ ID NO. 2. Numbering of amino acid positions is in reference to the mature S. mobaraensis Tgase sequence depicted in SEQ ID NO. 2. A “−” indicates a reduction in activity. A “+” indicates an improvement between 1.2 and 2-fold. A “++” indicates an improvement between 2 and 5-fold. A “+++” indicates an improvement greater than 5-fold.

In some embodiments, the Tgase variant comprises a combination of substitutions depicted in Table 6, further comprising one or more substitutions depicted in Table 3 and/or one or more substitutions depicted in Table 4.

In some embodiments, the Tgase variant has improved rate and/or productivity between 1.2 and 2-fold higher relative to wild-type mature Tgase amino acid sequence set forth in SEQ ID NO: 2. In some embodiments, the Tgase variant has improved rate and/or productivity between 2- and 5-fold higher relative to wild-type mature Tgase amino acid sequence set forth in SEQ ID NO: 2. In some embodiments, the Tgase variant has 1.2-fold, 1.3-fold, 1.4-fold, 1.5-fold, 1.6-fold, 1.7-fold, 1.8-fold, or 1.9-fold higher rate and/or productivity relative to wild-type mature Tgase. In some embodiments the Tgase variant has 2-fold, 2.5-fold, 3-fold, 3.5-fold, 4-fold, 4.5-fold, or 5-fold higher rate and/or productivity relative to wild-type mature Tgase.

In some embodiments, the Tgase variant comprises at least one or more amino acid substitutions with respect to SEQ ID NO: 2 comprising one of more of the following: substitution of A10 with T; substitution of D14 with W; substitution of R15 with T; substitution of Q51 with E; substitution of A166 with W; substitution of F170 with I; substitution of N282 with M; substitution of G283 with A; substitution of H289 with I; or substitution of S299 with A or V. In some embodiments, the Tgase variant comprises the substitutions in variant S1, S2, S3, S4, S5, S6, S7, S8, S9, S10, S11, S12, S13, S14, S15, S16, S17, S18, S19, S20, or S21 (Table 5). In some embodiments, the Tgase variant comprises the substitutions in variant S16.

In some embodiments, the Tgase variant comprises at least one or more amino acid substitutions with respect to SEQ ID NO: 2 comprising a combination of amino acid substitutions selected from: substitution of N282 with M and G283 with A; substitution of D14 with W, A166 with W, N282 with M, G283 with A, and S299 with V; substitution of N282 with M, G283 with A, and S299 with V; substitution of D14 with W, N282 with M, G283 with A, and S299 with A; substitution of N282 with M, G283 with A, and S299 with V; substitution of A10 with T, N282 with M, G283 with A, and S299 with V; substitution of D14 with W, N282 with M, G283 with A, and S299 with V; substation of R15 with T, N282 with M, G283 with A, and H289 with I; and substitution of Q51 with E, F170 with I, N282 with M, G283 with A, H289 with I, and S299 with V. In some embodiments, the Tgase variant comprises the substitutions in variant S1, S2, S3, S4, S5, S6, S7, S8, S9, S10, S11, S12, S13, S14, S15, S16, S17, S18, S19, S20, or S21 (Table 5). In some embodiments, the Tgase variant comprises the substitutions in variant S16.

In some embodiments, the Tgase variant comprises the following amino acid substitutions with respect to SEQ ID NO: 2: substitution of Q50 with S, Q74 with A, K91 with H, Q124 with L, K151 with R, K152 with R, N282 with M, and G283 with A. In some embodiments, the Tgase variant comprises the substitutions in variant S1, S2, S3, S4, S5, S6, S7, S8, S9, S10, S11, S12, S13, S14, S15, S16, S17, S18, S19, S20, or S21 (Table 5). In some embodiments, the Tgase variant comprises the substitutions in variant S16. In some embodiments, the Tgase variant comprises the sequence of SEQ ID NO: 4.

In some embodiments, the Tgase variant comprises the following amino acid substitutions with respect to SEQ ID NO: 2: substitution of D14 with W, Q50 with S, Q74 with A, K91 with H, Q124 with L, K151 with R, K152 with R, A166 with W, N282 with M, G283 with A, and S299 with V. In some embodiments, the Tgase variant comprises the substitutions in variant S1, S2, S3, S4, S5, S6, S7, S8, S9, S10, S11, S12, S13, S14, S15, S16, S17, S18, S19, S20, or S21 (Table 5). In some embodiments, the Tgase variant comprises the substitutions in variant S16. In some embodiments, the Tgase variant comprises the sequence of SEQ ID NO: 5.

In some embodiments, the Tgase variant has the sequence of SEQ ID NO: 3, further comprising one or more substitutions from Tables 2-4. In some embodiments, the Tgase variant has the sequence of SEQ ID NO: 4, further comprising one or more substitutions from Tables 2-4. In some embodiments, the Tgsae variant has the sequence of SEQ ID NO: 5, further comprising one or more substitutions from Tables 2-4.

The disclosed Tgase variants may comprise additional mutations or substitutions than those depicted in Tables 2-6. In some embodiments, the Tgase variant comprises one or more thermostability mutations. In some embodiments, the Tgase variant comprises a substitution of S2 (with respect to SEQ ID NO: 2) with an amino acid selected from C, D, E, F, H, I, K, L, M, N, P, Q, R, T, V, W, or Y. In some embodiments, the Tgase variant comprises a substitution of S2 with P with respect to the sequence of SEQ ID NO: 2. In some embodiments, the Tgase variant comprises no more than 15, 14, 13, 12, 11, or 10 amino acid substitutions compared to SEQ ID NO: 2. In some embodiments, the Tgase variant comprises no more than 15 amino acid substitutions compared to SEQ ID NO: 2.

In some embodiments, the Tgase variant comprises an N-terminal methionine residue, a C-terminal polyhistidine sequence, and/or a pro-sequence. The mature wild-type Streptomyces mobaraensis Tgase sequence lacks an N-terminal methionine. In some embodiments, the Tgase variant does not comprise an N-terminal methionine residue. In preferred embodiments, the Tgsae variant comprises an N-terminal methionine residue.

In some embodiments, the Tgase variant is expressed in the mature form (i.e., lacking a covalently attached a pro-peptide) with an N-terminal methionine residue. In some embodiments, the Tgase variant is expressed as a recombinant zymogen form of Tgase, wherein the Tgase variant zymogen comprises an N-terminal methionine and a methionine following the pro-peptide sequence. A recombinant zymogen form of Streptomyces mobaraensis Tgase with an N-terminal methionine and a methionine at the N-terminal of the mature sequence (i.e., immediately following the pro-peptide sequence) is shown in SEQ ID NO: 6. The pro-peptide sequence is bolded and underlined.

In some embodiments, the Tgase variant comprises a pro-peptide. One of skill in the art will recognize that amino acid substitutions disclosed herein that are described in reference to the wild-type mature Tgase sequence in SEQ ID NO: 2 will occur at corresponding residue in the mature sequence of the Tgase variant comprising a propeptide. The pro-peptide may be the naturally-occurring pro-peptide sequence for the mature Tgase, or it may be engineered to enhance pH-responsive, binding properties, productivity, and/or thermostability, among other qualities, as disclosed in U.S. 63/344,392. In some embodiments, the pro-peptide is the naturally occurring pro-sequence from the zymogen form of the Streptomyces mobaraensis Tgase (see bold, underlined text in SEQ ID NO: 1). In some embodiments, the pro-peptide comprises an N-terminal methionine. In some embodiments, the pro-peptide has the sequence of SEQ ID NO: 7.

In some embodiments the Tgase variant is expressed with a pro-sequence, either as part of the variant polypeptide sequence or as a separate polypeptide. In some embodiments, the mature variant polypeptide is expressed in the presence of a polypeptide Tgase pro-sequence. In some embodiments, a DNA sequence that encodes the pro-sequence and the DNA sequence that encodes the mature Tgase variant are expressed as discrete polypeptide sequences from the same DNA template. In some embodiments, the DNA sequence that encodes the mature polypeptide is expressed from a first DNA template, and the DNA sequence that encodes the pro-sequence is expressed from a separate second DNA template. In some embodiments, the pro-sequence is synthesized chemically and added to an expression system prior to, during, or after expression of the mature polypeptide.

In some embodiments, the Tgase variant comprises a C-terminal polyhistidine sequence. The C-terminal polyhistidine sequence may allow for isolation of recombinant Tgase through affinity chromatography. In some embodiments, the C-terminal polyhistidine sequence is a sequence of 10, 9, 8, 7, 6, 5, or 4 histidine residues. In some embodiments, the C-terminal polyhistidine sequence is a sequence of 8, 7, or 6 histidine residues. In some embodiments, the C-terminal polyhistidine sequence is a sequence of 6 histidine residues. In some embodiments, the Tgase variant comprises a linker of 1 to 5 amino acids between immediately 5′ to the C-terminal polyhistidine sequence. In calculating percent identity between the Tgase variant and a reference sequence, or the number of different amino acids between the Tgase variant and a reference sequence, the C-terminal polyhistidine sequence is not taken into consideration.

In some embodiments, one or more of the glutamine residues in the sequence of a Tgase variant may be substituted with a glutamate residue as compared to SEQ ID NO: 2. As discussed above, Tgases undergo self-deamination of glutamine residues, which may result in the hydrolysis of glutamine residues to glutamate. As such, Tgase variants having one or more glutamine residues replaced with glutamate as compared to SEQ ID NO: 2 are contemplated herein.

In some embodiments, the Tgase variant comprises a sequence having at least about 80%, 85%, 90%, 91%, 92%, 93%, 94%, 95%, 96%, 97%, 98%, 99%, or 99.5% sequence identity in comparison to the wild-type mature Tgase amino acid sequence set forth in SEQ ID NO: 2. In some embodiments, the Tgase variant comprises a sequence having at least 90% sequence identity the SEQ ID NO: 2. In some embodiments, the Tgase variant comprises a sequence having at least 95% sequence identity to SEQ ID NO: 2.

In some embodiments, the Tgase variant comprises a sequence having at least about 80%, 85%, 90%, 91%, 92%, 93%, 94%, 95%, 96%, 97%, 98%, 99%, or 99.5% sequence identity in comparison to the Tgase variant amino acid sequence set forth in SEQ ID NO: 3, SEQ ID NO: 4, or SEQ ID NO: 5. In some embodiments, the Tgase variant comprises a sequence having at least 90% sequence identity to SEQ ID NO: 3, SEQ ID NO: 4, or SEQ ID NO: 5. In some embodiments, the Tgase variant comprises a sequence having at least 95% sequence identity to SEQ ID NO: 3, SEQ ID NO: 4, or SEQ ID NO: 5.

In some embodiments, the Tgase variant comprises no more than 20, 19, 18, 17, 16, 15, 14, 13, 12, 11, 10, 9, 8, 7, 6, 5, 4, 3, 2, or 1 amino acid substitutions compared to SEQ ID NO: 2. In some embodiments, the Tgase variant comprises no more than 15, 14, 13, 12, 11, or 10 amino acid substitutions compared to SEQ ID NO: 2. In some embodiments, the Tgase variant comprises no more than 15 amino acid substitutions compared to SEQ ID NO: 2. In some embodiments, the Tgase variant comprises no more than 13 amino acid substitutions compared to SEQ ID NO: 2. In some embodiments, the Tgase variant comprises no more than 20, 19, 18, 17, 16, 15, 14, 13, 12, 11, 10, 9, 8, 7, 6, 5, 4, 3, 2, or 1 amino acid substitutions compared to SEQ ID NO: 3, SEQ ID NO: 4, or SEQ ID NO: 5. In some embodiments, the Tgase variant comprises no more than 15, 14, 13, 12, 11, or 10 amino acid substitutions compared to SEQ ID NO: 3, SEQ ID NO: 4, or SEQ ID NO: 5. In some embodiments, the Tgase variant comprises no more than 13 amino acid substitutions compared to SEQ ID NO: 3, SEQ ID NO: 4, or SEQ ID NO: 5. In some embodiments, the Tgase variant comprises no more than 11 amino acid substitutions compared to SEQ ID NO: 3, SEQ ID NO: 4, or SEQ ID NO: 5.

In some embodiments, the Tgase variant comprises no deletions or truncations compared to SEQ ID NO: 2. In some embodiments, the Tgase variant comprises no additions compared to SEQ ID NO: 2, other than optionally an N-terminal methionine and/or a C-terminal polyhistidine sequence.

### Compositions

Disclosed are compositions, e.g., biocidal, preservative, antimicrobial, antibacterial, and antiviral (virucidal) compositions that include one or more Tgase variants having aseptilase activity. Also disclosed are compositions, e.g., biocidal, preservative, antimicrobial, antibacterial, and antiviral compositions that include one or more Tgase variant as disclosed herein. Such a composition may be included in or with (e.g., within or associated with) products to be preserved, e.g., for microbial control. The Tgase variant may catalyze a reaction of amino acid residues on a protein, thereby effecting, for example, protein crosslinking or binding a molecule of interest to a protein. In some embodiments, the Tgase variant is incorporated into the composition in an amount effective to prevent or decrease growth of one or more microbe in comparison to the identical composition that does not comprise the variant. In some embodiments, the compositions include one or more Tgase variant, e.g., comprising or consisting of one or more Tgase variants as disclosed herein, in an amount effective to inhibit microbial (e.g., bacterial) growth, resulting in inhibition of up to at least 80%, 85%, 90%, 95%, 98%, 99%, 99.9%, 99.99%, or 99.999% of microbial growth.

In some embodiments, the compositions include one or more additional antimicrobial proteins or enzymes, such as a crosslinking enzyme, oxidase, nuclease, hydrolase, protease, and/or lytic enzyme. In some embodiments, the composition includes one or more antimicrobial chemicals, such as, but not limited to, sodium benzoate, phenoxyethanol, coco betaine, and glycols (e.g. caprylyl glycol, hexanediol, propane diol, ethylhexylglycerin, etc.) Exemplary, but nonlimiting examples of antimicrobial enzymes, compositions, and formulations, and methods of use thereof, are disclosed in WO 2020/181099, which is incorporated by reference herein in its entirety. In some embodiments, the composition includes both one or more additional antimicrobial enzymes and one or more antimicrobial chemicals.

Enzyme compositions are disclosed that contain the Tgase variant in compositions used for long-lasting applications including personal care, leather, textiles, household, industrial, food, pharmaceutical, cosmetic, healthcare, marine, paint, coating, energy, plastic, packaging, or agricultural products. The disclosed compositions include one or more Tgase variant for the purpose of reacting amino acid residues on skin or hair protein or other protein-, peptide-, or amino acid-containing materials with a molecule of interest, such as a sunscreen, or color producing molecule, e.g., a pigment or dye. In some embodiments, the Tgase enzyme acts as a catalyst of a linker with an amino acid (e.g., side chain of glutamine or lysine residues) in skin, hair, or nail proteins or peptides.

In some embodiments, a variant or wild-type Tgase enzyme can be used to create a permanent or semipermanent film on the skin, hair, or nails with a second peptide or protein. These methods may be used to create films that reduce color transfer, maintain color on the surface, and improve visual or structural properties of the skin, hair, or nails, as compared to untreated materials. Some embodiments include methods of reducing frizz or retaining style in hair, or reducing fraying, peeling, or wrinkling in textiles by treating an object with a variant or wild-type Tgase. These improvements can also be applied to cosmetic applications, textiles, or leather. In some embodiments, improvements in moisture resistance of the fiber (skin, hair, nails, textile, or leather) can be observed, such as improving water resistance of leather or textiles. In some embodiments, improvements in the strength or durability of the fiber is observed upon addition of the Tgase catalyzed permanent film. For these applications, a variant Tgase may be used in lesser amounts than a wild-type Tgase. For example, a variant Tgase may be applied to a textile as a composition comprising about 10 to about 10,000 ppm. In some embodiments, a composition comprising about 500 ppm of a transglutaminase is applied to the leather for repairing, coating, and/or improving water resistance of leather.

In some embodiments, a variant or wild-type Tgase enzyme can be used for tanning hide. For example, the tanning process may comprise applying to the hide a transglutaminase at about 0.01% to about 5% w/w relative to the weight of the hide.

### A. Antimicrobial Proteins and Peptides

In some embodiments, the compositions include a disclosed Tgase variant and one or more antimicrobial peptides. In some embodiments, the compositions include a Tgase variant having aseptilase activity and one or more antimicrobial peptides. Examples of antimicrobial peptides include, but are not limited to, nisin and pediocin.

In some embodiments, the compositions include a disclosed Tgase variant and one or more additional antimicrobial proteins or enzymes. In some embodiments, the compositions include a Tgase variant having aseptilase activity and one or more antimicrobial proteins or enzymes. Examples of antimicrobial proteins include, but are not limited to, lysozyme.

Nonlimiting examples of known antimicrobial or antimicrobial proteins, enzymes, and peptides, which may be included in combination with a Tgase variant as disclosed herein, are shown in Table 7. In some embodiments, a Tgase variant as described herein may be utilized in an antimicrobial, preservative, antibacterial, or antiviral (virucidal) composition in combination with one or more of the antimicrobial enzymes, peptides, or proteins described in Table 7.

TABLE 7
Enzymes, Peptides, and Proteins with Known Antimicrobial Properties

| Mechanism | Enzyme | Description | Citation |
|---|---|---|---|
| Lytic | Lysozyme | Produced by animals as | Ibrahim et al. (2001) |
| | | part of the innate immune | FEBS Letters 506(1): 27- |
| | | system. Hydrolyzes the | 32; Małaczewska et al. |
| | | peptidoglycan subunits in | (2019) BMC Vet. Res. |
| | | the bacterial cell wall. | 15: 318 |
| | Chitinase | Secreted by soil bacteria | Martínez-Zavala et al |
| | | including Bacillus | (2020) Front. Microbiol. |
| | | thuringiensis to combat | 10: 3032 |
| | | insects and fungi | |
| | Lipase | Hydrolyzes extracellular | Prabhawathi et al. (2014) |
| | | lipids and polymers. | PLoS One 9(5) |
| | Lysin | Utilized by bacteriophages | Hoops et al. (2008) Appl. |
| | | to hydrolyze the glycan | Environ. Microbiol. 75: 5, |
| | | component of bacterial cell | 1388-1394 |
| | | wall | |
| | Lysostaphin | Metalloendopeptidase | Kokai-Kun et al. (2003) |
| | | which cleaves the | Antimicrob Agents |
| | | pentaglycine bridges found | Chemother 47(5): 1589- |
| | | in cell wall peptidoglycan. | 1597 |
| | Glucanase | Secreted by soil bacteria | Shafi et al. (2017) |
| | | including Bacillus species | Biotechnology & |
| | | to degrade the fungal cell | Biotechnological |
| | | wall. Has also been utilized | Equipment 31: 3 446-459 |
| | | as an algicide and for | |
| | | biofilm control. | |
| Nuclease | DNase | Hydrolyzes extracellular | Kaplan et al. (2012) J. |
| | | nucleic acids and viral | Antibiot. (Tokyo) |
| | | genomic DNA. | 65(2): 73-77 |
| | RNase | Hydrolyzes viral RNA. | Wirth (1992) |
| | | | WO1994000016A1 |
| | Lactoferrin | Sequesters essential iron | Niaz et al. (2019) |
| | | ions to prevent microbial | International Journal of |
| | | growth. Also possesses | Food Properties 22: 1 |
| | | nuclease activity and | 1626-1641 |
| | | hydrolyzes biofilm | |
| | | polymers. | |
| Oxidoreductase | Glucose Oxidase | Oxidizes glucose to D- | Wong et al. (2008) Appl |
| | | glucono-δ-lactone and | Microbiol Biotechnol. |
| | | hydrogen peroxide. | 78(6): 927-938 |
| | Peroxidase | Oxidizes inert substrates to | Ihalin et al. (2006) Arch. |
| | | form antimicrobial actives. | Biochem. Biophys. 445, |
| | | | 261-268 |
| | Lactoperoxidase | Oxidizes inert substrates to | White et al. (1983) |
| | | form antimicrobial actives. | Antimicrob Agents |
| | | | Chemother 32(2): 267-272 |
| Quorum | Lactonase | Hydrolyzes quorum | Schwab et al. (2019) |
| Quenching | | sensing lactones, | Front Microbiol. 10: 611 |
| | | preventing activation of | |
| | | biofilm- and pathogenesis- | |
| | | promoting pathways. | |
| | Acylase | Hydrolyzes quorum | Vogel et al. (2020) Front. |
| | | sensing lactones, | Chem. 8: 54 |
| | | preventing activation of | |
| | | biofilm- and pathogenesis- | |
| | | promoting pathways. | |
| Hydrolase | Dispersin B | Hydrolyzes biofilm | Izano et al. (2007) J Dent |
| | | polymers | Res 86(7): 618-622 |
| | α-amylase | Hydrolyzes extracellular | Craigen et al. (2011) Open |
| | | polysaccharides. | Microbiol J. 5: 21-31 |
| | Cellulase | Hydrolyzes the cellulose | Loiselle et al. (2003) |
| | | component of biofilms and | Biofouling 19(2): 77-85 |
| | | algal cell walls. | |
| Antimicrobial | Nisin | Increases permeability of | Li et al. (2018) Appl |
| Peptides | | the microbial cell | Environ Microbiol 18(12) |
| | | membrane. | |
| | Bacteriocin | Modes of action include | Meade et al. (2020) |
| | | inhibition of cell wall | Antibiotics 9(1): 32 |
| | | synthesis and increasing | |
| | | cell membrane | |
| | | permeability. | |
| | Siderophore | Binds to and sequesters | Raaska et al. (1999) J |
| | | iron ions | Indust Microbiol |
| | | | Biotechnol 22, 27-32 |
| | Polymyxin | Increases permeability of | Poirel et al. (2017) Clin |
| | | the microbial cell | Microbiol Rev 30: 577-596 |
| | | membrane. | |
| | Defensin | Increases permeability of | Gans (2003) Nat Rev |
| | | the microbial cell | Immunol 3, 710-720 |
| | | membrane. | |

### B. Antimicrobial Chemicals

In some embodiments, a Tgase variant as described herein may be formulated with one or more antimicrobial chemical, including, but not limited to sodium benzoate, phenoxyethanol, glycols, or quaternary ammonium compounds, for example, for use as an antimicrobial, preservative, antibacterial, or antiviral (virucidal) composition. In some embodiments, a Tgase variant having aseptilase activity may be formulated with one or more antimicrobial chemicals. Nonlimiting examples of antimicrobial chemicals are shown in Table 8.

TABLE 8
Examples of Antimicrobial Chemicals

| Classification | Chemical |
|---|---|
| Polymers | Chitosan |
| | N,N,N-trimethyl chitosan |
| | ε-poly-lysine |
| | Polyvinylbenzyl-dimethylbutyl ammonium chloride |
| | Polyvinylbenzyl trimethyl ammonium chloride |
| | Quaternary ammonium polyethyleneimine |
| | Quaternary phosphonium modified epoxidized |
| | natural rubber |
| | Arginine-tryptophan-rich peptide |
| | Guanylated polymethacrylate |
| | Ammonium ethyl methacrylate homopolymers |
| | Metallo-terpyridine carboxymethyl cellulose |
| | Poly(n-vinylimidazole) modified silicone rubber |
| | Ammonium containing cationic polymers |
| Quaternary | Cocamidopropyl Betaine |
| Ammonium | Myristamidopropyl-pg-dimonium Cl Phosphate |
| | Benzalkonium Chloride (BZK) |
| | Quaternium-6 |
| | Lauryl Betaine |
| | Coco Betaine |
| Detergents | Sodium Lauryl Sulfate |
| | Dodecylbenzenesulfonic Acid |
| Chaotropic Agent | Polyamidopropyl biguanide |
| | Guanidinium chloride |
| Organic Acids | Lactic Acid |
| | Citric Acid |
| | Salicylic Acid |
| | Sorbic Acid (Potassium/Calcium Sorbate) |
| | Acetic Acid |
| | Dehydroacetic Acid |
| | Ascorbic acid |
| | Caprylic acid |
| | Benzoic Acid (Sodium Benzoate) |
| Phenols, | Ethanol |
| Alcohols, & | Isopropanol |
| Polyols | Dichlorobenzyl Alcohol |
| | Glycerol |
| | Caprylyl Glycol |
| | Ethylhexylglycerin |
| | 1,3-Propanediol |
| | 1,2-Hexanediol and 1,6-Hexanediol |
| | 1,2-Pentanediol |
| | 1,10-Decanediol |
| | 2,3-Butanediol |
| | Phenylpropanol |
| | Caprylyl glyceryl ether |
| | Gluconolactone |
| | Hydroxyacetophenone |
| | Benzyl Alcohol |
| | Phenethyl alcohol |
| | p-Anisic acid |
| | 2-Phenoxyethanol |
| Aldehydes & | Glutaraldehyde |
| Aldehyde | Formaldehyde and Formaldehyde releasers |
| Releasers | Sodium Hydroxymethylglycerate |
| | DMDM Hydantoin |
| Base | Sodium Hydroxide |
| Oxidizers | Hydrogen Peroxide and Peracids (peracetic acid) |
| Chelators | Ethylenediaminetetracetic acid (EDTA) |
| | tetrasodium glutamate diacetate (GLDA) |
| | methylglycine N,N-diacetic acid (MDGA) |
| Parabens | Methyl Paraben |
| | Ethyl Paraben |
| | Propyl Paraben |
| Miscellaneous | Natamycin |
| | Benzisothiazolinone (BIT) |
| | Bronopol |
| | Sorbitan Caprylate |
| | Ethyl Lauroyl Arginate |
| | Methylisothiazolinone (MIT) |
| | Cetylpyridinium Chloride |
| | Chlorphenesin |
| | Zinc Omadine |
| | Sodium Omadine |
| | N-(3-aminopropyl)-N-dodecylpropane-1,3-diamine |
| | Methylchloroisothiazolinone |
| | 2,2-dibromo-3-nitrilopropionamide |
| | 1-Octadecanaminium, N,N-dimethyl-N-[3- |
| | (trimethoxysilyl)propyl]-, chloride |
| | Saponin |
| | Ethyl lauroyl arginate |
| | Capryl hydroxamic acid (CHA) |
| Cell | Polymyxin B, chlorohexidine, hexametaphosphate, |
| permeabilizers | and phytic acid |

Other miscellaneous antimicrobial chemicals include: iodopropynyl butylcarbamate (IPBC), polyhexamethylene biguanide (PHMB), 1,2-dibromo-2,4-dicyanobutane (DBDCB), and Styrene acrylates.

### Products and Uses

In some embodiments, products disclosed herein include personal care products, household products, industrial food, pharmaceutical, cosmetic, healthcare, marine, paints, coatings, adhesives, energy, plastic, packaging, or agricultural products, optionally immobilized on or encapsulated in a polymeric support, which include an effective amount, for example, about 0.00001% w/v to about 5% w/v, or, for example, 0.0001% w/v to about 5% w/v, of Tgase variants as described herein, or a composition thereof as described herein, to act as an antimicrobial agent, e.g., preservative, in the product. Examples of polymeric supports for use with Tgase enzymes and variants are disclosed in WO 2020/1231705, which is incorporated by reference herein.

In some embodiments, products disclosed herein include cosmetics and personal care products, which include compositions described herein, compositions that include one or more Tgase variant, and one or more active or functional ingredients which may include a sunscreen and/or color producing molecule, in an amount effective to bond a sunscreen molecule or color to a surface, such as covalently binding to one or more proteins or peptides either on the surface of skin or within the product formulation, e.g., hydrolyzed protein, collagen, keratin, and/or elastin. In some embodiments, the product composition includes any of the Tgase variants disclosed in SEQ ID NOs: 1-7. In some embodiments, an effective amount of Tgase variants is in a range of about 0.00001% to about 5%, about 0.0001% to about 5% w/v, about 0.001% to about 1%, or about 0.01% to 0.1%, by weight of the composition. In some embodiments, the active or functional ingredient (e.g., protein, peptide, sunscreen molecule, or coloring agent) is present in the composition in an amount effective to provide a benefit, such as frizz control, color retention, color transfer reduction, shine improvement, moisture resistance, strengthening, a UV protecting benefit or a cosmetic or aesthetic benefit, e.g., to hair, skin, or nail proteins or peptides or to the hair, skin, or nails of an individual to whom the composition is topically applied. In some embodiments, the effective amount of the active or functional ingredient (e.g., sunscreen molecule or coloring agent) is in a range of about 0.1% to about 70%, such as about 1% to about 35%, by weight of the composition.

In some embodiments, products disclosed herein include cosmetics, skincare, haircare, or personal care products which include compositions described herein, compositions that include a Tgase variant and one or more proteins or peptides which when applied by the consumer can covalently bind to either on the surface of skin, hair, or nails.

In some embodiments, products disclosed herein include leather products, animal hides, vegan leather, textiles, or clothing which include compositions described herein, compositions that include a Tgase variant covalently binding one or more proteins or peptides either on the surface of the product creating a permanent film or coating on the product.

In some embodiments, the Tgase variant as described herein is included in a personal care product, such as, but not limited to, bar soap, liquid or hand soap, hand sanitizer (including rinse off and leave-on alcohol based and aqueous-based hand disinfectants), preoperative skin disinfectant, cleansing wipes, disinfecting wipes, body wash, acne treatment products, antifungal diaper rash cream, antifungal skin cream, shampoo, conditioner, cosmetics (including but not limited to liquid or powder foundation, liquid or solid eyeliner, mascara, cream eye shadow, tinted powder, “pancake” type powder to be used dry or moistened, make up removal products, etc.), deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. In some embodiments, the Tgase variant is included in a wound care item, such as, but not limited to, wound healing ointments, creams, and lotions, wound coverings, burn wound cream, bandages, tape, and steri-strips, and medical articles such as medical gowns, caps, face masks, and shoe-covers, surgical drops, etc. In some embodiments, the Tgase variant is included in an oral care product, such as mouth rinse, toothpaste, or dental floss coating, a veterinary or pet care product, a preservative composition, or a surface disinfectant, such as a disinfectant solution, spray, or wipe.

In some embodiments, the Tgase variant is in a product, such as a personal care, household, industrial, food, pharmaceutical, cosmetic, healthcare, marine, paint, coating, adhesive, energy, plastic, packaging, or agricultural product, or in any of the products or systems disclosed herein, microbial growth is decreased, the product is preserved, and/or shelf life of the product is increased in comparison to an identical product that does not contain the Tgase variant.

In some embodiments, a method for increasing the shelf life, integrity, or microbial free (e.g., bacterial and/or fungal free) status of a product composition, or preserving a product composition, such as a personal care, household or industrial product is provided, wherein the method includes incorporating an effective amount of the Tgase variant in the composition as disclosed herein into the product. In some embodiments, the effective amount may be an amount, referred to as the MIC (minimum inhibitory concentration), which results in reduction of microbial growth by approximately 80-100%, or any of at least about 80%, 85%, 90%, 95%, 98%, 99%, 99.9%, 99.99%, or 99.999% reduction of microbial growth as described herein.

In some embodiments, the Tgase variant, such as any of the variants disclosed herein, or a composition thereof as disclosed herein, is included as an antimicrobial agent in any of the products disclosed herein at a concentration of any of at least about 0.00001% w/v, 0.0001% w/v, 0.0005% w/v, 0.001% w/v, 0.005% w/v, 0.01% w/v, 0.05% w/v, 0.1% w/v, 0.5% w/v, 1% w/V, 1.5% w/v, 2% w/v, 2.5% w/v, 3% w/v, 3.5% w/v, 4% w/v, 4.5% w/v, or 5% w/v. In some embodiments, the Tgase variant comprises a composition thereof included at a concentration of any of about 0.00001% w/v to about 0.005%, 0.0001% w/v to about 0.0005% w/v, about 0.001% w/v to about 0.005% w/v, about 0.005% w/v to about 0.01% w/v, about 0.01% w/v to about 0.05% w/v, about 0.05% w/v to about 0.1% w/v, about 0.1% w/v to about 0.5% w/v, about 0.5% w/v to about 1% w/v, about 1% w/v to about 1.5% w/v, about 1.5% w/v to about 2% w/v, about 2% w/v to about 2.5% w/v, about 2.5% w/v to about 3% w/v, about 3% w/v to about 3.5% w/v, about 3.5% w/v to about 4% w/v, about 4% w/v to about 4.5% w/v, about 4.5% w/v to about 5% w/v, about 0.00001% w/v to about 0.0001% w/v, about 0.0001% w/v to about 0.001% w/v, about 0.001% w/v to about 0.01% w/v, about 0.01% w/v to about 0.1% w/v, about 0.1% w/v to about 1% w/v, about 1% w/v to about 2.5% w/v, about 2.5% w/v to about 5% w/v, or about 1% w/v to about 5% w/v.

Non-limiting examples of household/industrial products using the disclosed Tgase variants in compositions thereof, include householder cleaners such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. used in a food wash product, designed to clean fruits and vegetables prior to consumption, packaging, and food coatings.

In some embodiments, one or more Tgase variants disclosed herein, or a composition thereof, is used for methods of cleaning in place (CIP). CIP methods disclosed herein can be used for manufacturing equipment and/or pipelines used to manufacture products disclosed herein, household products, cosmetics, industrial products, pharmaceutical products, biological products, food and beverage products and the like. CIP methods disclosed herein can also be used in biotechnology processes.

In some embodiments, one or more disclosed Tgase variants, or a composition thereof, is added directly to manufacturing streams to maintain sanitary conditions and/or reduce microbial load in processing steps. In some embodiments, the Tgase variant added to the manufacturing streams is either not present at a significant level in the final product or has no functional or technical effect in the final product. In some embodiments, one or more disclosed Tgase variants, or a composition thereof, is used for decontamination or cleaning of raw materials, final products, and/or waste streams. In some embodiments, one or more disclosed Tgase variants, or a composition thereof, is used for reducing microbial load or bioburden in manufacturing streams, raw materials, final products, and/or waste streams. An example of decontamination of a product (hand soap) using a disclosed Tgase variant is provided in Example 10.

### EXAMPLES

The following examples are intended to illustrate, but not limit, the invention. Accordingly, from the above discussion and the Examples, one skilled in the art can ascertain essential characteristics of this disclosure, and without departing from the spirit and scope thereof, can make various changes and modifications to adapt to various uses and conditions.

Unless defined otherwise herein, all technical and scientific terms used herein have the same meaning as commonly understood by one of ordinary skill in the art to which this disclosure belongs.

### Example 1. Tgase Vector Construction and Mutagenesis

### A. Cell Free Protein Synthesis (CFPS) Vectors

The gene coding for mature Tgase was codon optimized for expression in E. coli based on the published amino acid sequence (Kanaji, et al. (1993) J. Biol. Chem.; 268 (16): 11565-11572), synthesized, and cloned onto a pUC19-derived expression vector as described in PCT/US20/49226.

B. E. coli Vector

The gene coding for the Pro-Tgase variant (i.e., zymogen form of Tgase variant) was codon optimized for expression in E. coli based on the published amino acid sequence (Kanaji, et al. (1993) J. Biol. Chem.; 268 (16): 11565-11572), synthesized with an additional C-terminal His tag, and cloned into a pET vector operatively linked to the T7 promoter. The expression vector also contains the pMB1 origin of replication and a kanamycin resistance gene. The resulting plasmid was transformed first into E. coli DH-10B, using standard methods known in the art. The transformants were isolated by subjecting the cells to kanamycin selection, as known in the art (See, e.g., U.S. Pat. No. 8,383,346 and WO 2010/144103, both of which are incorporated by reference herein, in their entirety), and the sequence of the Pro-Tgase gene was verified by Sanger sequencing. The plasmid was recovered from a positive clone, using methods known in the art, and transformed into E. coli BL21 (DE3) for expression.

### C. Creation of Tgase Variants

Mutations were introduced into the Pro-Tgase and mature Tgase genes described in Examples 1A and 1B using a combination of de novo DNA synthesis as described in PCT/US20/49226 and site directed mutagenesis methods known in the art.

### Example 2. Expression and Screening of Tgase Variants Using Cell-Free Protein Synthesis

### A. Cell Free Protein Synthesis

Mature Tgase variants were expressed in a commercially available cell-free protein synthesis kit following the manufacturer's instructions as described in WO 2021/178001. Following mature Tgase expression, Tgase activity was assayed as described in Example 4.

### B. Screening of Mature Tgase Variants

To evaluate chemostability, Tgase samples in cell-free extract were assayed for activity, using the assay described in Example 4B, immediately after expression and again following a 24 hour incubation at 20-30° C. Mature Tgase variant residual activity was normalized to the residual activity of the wild-type and results are depicted in Table 5.

To evaluate productivity, total product formed was measured after 24-hours of incubation, using the assay described in Example 4B. The time is defined as 24 hours which represents approaching or surpassing burnout, denaturation, or self-reaction of the enzyme variant. Results are depicted in Table 6.

Rate was assessed by measuring the rate of product formation over 60 minutes of incubation using a kinetic method to take scans every 60 seconds to determine product formation, using reaction conditions as described in Example 4B. The enzyme produces product at an initial rate that is approximately linear for the first one hour of the reaction. The concentrations of enzyme and substrate are the same across the sample set for each enzyme variant. Enzyme variants with faster initial rates deliver a higher product yield in one hour than variants with slower initial rates produce at the same time-period. Results are depicted in Table 6.

### Example 3. Characterization of a Pro-Tgase Variant

A. Expression and Isolation of Recombinant Pro-Tgase Variant (SEQ ID NO:6) in E. coli

The E. coli strain BL21 (DE3), containing the Pro-Tgase expression vector described in Example 1C, was cultured overnight in Luria broth at 30-37° C. until the culture reached saturation. The following morning, the culture was used to inoculate a shake flask containing a medium including glycerol, soy peptone, yeast extract, magnesium sulfate heptahydrate, and potassium phosphate monobasic, followed by incubation at 30-34° C. for up to 10 hours with continuous shaking. Isopropyl β-d-1-thiogalactopyranoside (IPTG) was added to a final concentration of 0.1-1 mM and incubation was continued at 20-25° C. for up to 24 hours.

Cells were harvested by centrifugation at 8000× g for up to 60 minutes. The supernatant was discarded, and the pellet was resuspended to 20% w/v in 50 mM phosphate buffer, pH 7.4. The cells were lysed using a high-pressure homogenizer at pressures from 10000-15000 psi. The crude lysates, together with a flocculating agent added at 0.5-2.0% vol, were clarified through centrifugation at 15000×g for up to 60 minutes. The clarified lysate containing Pro-Tgase was assessed by sodium dodecyl sulfate polyacrylamide gel electrophoresis (SDS-PAGE) as described in Example 4C and by spectroscopy. After confirming the presence of Pro-Tgase, in the clarified lysate, the sample was concentrated and buffer exchanged against a solution of 50 mM phosphate buffer pH 7.4, 100 mM sodium chloride on a 10 to 50 kDa membrane.

Alternatively, the Pro-Tgase variant may be secreted, for example, from a microbial strain that is known to those skilled in the art to secrete Tgase such as Streptomyces mobaraensis or Bacillus subtilis.

Following secretion, cells are discarded via centrifugation and the supernatant is recovered and is assessed by SDS-PAGE as described in Example 4C and by spectroscopy. After confirming the presence of Pro-Tgase in the supernatant, the sample is concentrated and buffer exchanged against a solution of 50 mM phosphate buffer pH 7.4, 100 mM sodium chloride on a 10 to 50 kDa membrane.

### B. Proteolytic Activation of a Pro-Tgase Variant (SEQ ID NO: 6)

The semi-purified zymogen was activated as described in PCT/US2022/076202. A Solution containing Pro-Tgase was concentrated to at least 10 g/L Pro-Tgase by tangential flow filtration on a 10 to 50 kDa membrane and buffer exchanged into a solution of 50 mM phosphate buffer pH 7.4, 100 mM sodium chloride on a 10 to 50 kDa membrane. Following buffer exchange, glycerol and ammonium sulfate were added to final concentrations of 20% and 10 mM respectively, and then soluble transglutaminase-activating M4 metalloprotease (TAMEP) was added at 1% vol to the Pro-Tgase variant solution. The reactions were then incubated at 37° C. with agitation at 300 rpm. After incubating with soluble TAMEP for 15-120 minutes, soluble Streptomyces mobaraensis transglutaminase-activating tripeptidyl aminopeptidase (SM-TAP) was added to the reaction at 1% vol. The reactions were then incubated at 37° C. with agitation at 300 rpm for an additional 15-120 minutes Following activation, ethylenediaminetetraacetic acid (EDTA) and additional glycerol were added to final concentrations of 1 mM and 30%, respectively, and Tgase activity was assessed using the Colorimetric Activity Assay described in Example 4A.

### C. Storage Buffer of Mature Tgase Variants

Purified mature Tgase variants were stored at a concentration of 10 g/L in 30% glycerol, 50 mM phosphate buffer pH 7.4, 100 mM sodium chloride, 10 mM ammonium sulfate, and 1 mM EDTA.

### Example 4. Enzyme Assays

### A. Colorimetric Activity Assay

Tgase activity was measured herein using a colorimetric hydroxamate activity assay (Folk and Cole (1965) J Biol Chemistry 240 (7): 2951-2960). Briefly, the hydroxamate assay uses N-benzyloxycarbonyl-L-glutaminyl-glycine (ZQG) as a low molecular weight amine acceptor substrate and hydroxylamine as an amine donor. In the presence of catalytically active Tgase, the hydroxylamine is incorporated to form Z-glutamylhydroxamate-glycine, which develops a colored complex with iron (III), detectable at 525 nm after incubation at 37° C. for 5-200 minutes. The calibration was performed using L-glutamic acid gamma-monohydroxamate (Millipore® Sigma®) as a standard. One unit of Tgase was defined as the amount of enzyme that catalyzes formation of 1 μmol of the peptide derivative of gamma-glutamylhydroxylamine per minute. Activity of Tgase variants is depicted in Tables 2-4.

### B. Protein Functionalization Assay

Protein functionalization by Tgase variants was determined by the fluorogenic labeling of casein using monodansylcadaverine (e.g., a commercially available kit such as the Transglutaminase Fluorogenic Activity Assay Kit, T036, Zedira, Germany). Tgase activity was monitored by measuring fluorescence (excitation wavelength 332 nm; emission wavelength 500 nm) using a BioTek Synergy H1 microplate reader. Transglutaminase-catalyzed covalent coupling (crosslinking) of monodansylcadaverine with N,N-dimethylcasein produces a product that causes a shift in intensity and wavelength of fluorescence of the dansyl group now linked to the casein. The relative transglutaminase activity is shown by increase of fluorescence intensity over time. Enzyme variant loading was optimized based on expression levels, ensuring enzyme was the rate limiting factor. Briefly, cell-free reactions (set up as described in Example 2A) were first diluted 10-fold using phosphate buffer, pH 7.4, and a 5 microliter aliquot was analyzed.

### C. Sodium Dodecyl Sulfate Polyacrylamide Gel Electrophoresis (SDS-PAGE)

This technique was used to analyze the molecular weight of expressed proteins. Mature Tgase was secured from commercial sources (Moo Gloo TI formula, Ajinomoto®) and used as a molecular weight standard for SDS-PAGE analysis.

A 10 mg aliquot of the expressed protein was added to Novex™ Bolt™ LDS Sample Buffer and Novex™ Bolt™ Sample Reducing Agent, mixed as directed, heated at 95° C. for 5 minutes, and then run on a 4-12% SDS-PAGE gel (Invitrogen™ NuPAGE™ 4-12% Bis-Tris Gel) for 30 minutes at 170 volts. Gels were stained with SimplyBlue™ Safe Stain (Invitrogen™) for 30-60 minutes before being destained in deionized water and photographed.

### Example 5. Aseptilase Activity of a Tgase Variant by MIC Testing

E. coli (ATCC 8739) and C. albicans (ATCC 10231) were acquired from the American Type Culture Collection (ATCC) (Manassas, VA) and maintained as −80° C. frozen glycerol stocks.

For MIC determination of bacterial cultures, E. coli (ATCC 8739) was grown overnight (16-18 hours) in Mueller Hinton broth at 37° C. The following day, the cell density of the saturated cultures was calculated using OD₆₀₀ and cultures were diluted to 104 to 10⁶ CFU/mL in sterile Mueller Hinton broth to generate the inoculum, and 100 μL of the inoculum was combined with 100 μL of serially diluted Tgase variant A3 (SEQ ID NO: 4) at a range of 0.0001-0.05 weight percent. Growth curves were measured by OD₆₀₀ on a BioTek® Synergy Plate Reader. All test conditions were performed in triplicate. MIC was determined to be 200 ppm (0.005% w/v) against E. coli for the enzyme described in SEQ ID NO: 4 using OD₆₀₀. MIC was outside the range of detection against E. coli for the mature form of the enzyme described in SEQ ID NO: 6 using OD₆₀₀ (as described in Example 3).

For MIC determination of yeast cultures, C. albicans (ATCC 10231) was grown overnight (24 hours) in Sabouraud Dextrose broth at 30° C. The following day, the cell density of the saturated cultures was calculated using OD₆₀₀ and cultures were diluted to 104 to 10⁶ CFU/mL in sterile Sabouraud Dextrose broth media to generate the inoculum, and 100 μL of the inoculum was combined with 100 μL of serially diluted Tgase variant at a range of 0.0001-0.05 weight percent. The cultures were grown overnight at 30° C. and growth curves were measured by OD₆₀₀ on a BioTek® Synergy Plate Reader. All test conditions were performed in triplicate. MIC was determined to be 100 ppm (0.005% w/v) against C. albicans for the enzyme described in SEQ ID NO: 4 using OD₆₀₀. MIC was determined to be 25 ppm (0.0025% w/v) against C. albicans for the mature form of the enzyme described in SEQ ID NO: 6 using OD₆₀₀ (as described in Example 3).

### Example 6. Comparing Activity and Stability of Commercial Wild-Type Toase and a Tgase Variant

### A. Specific Activity

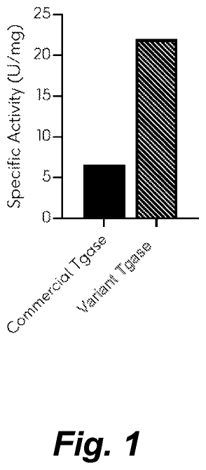

A commercially available wild-type Tgase (Ajinimoto®, Activa® TI formulation) formulated at 0.75-1.0% enzyme in maltodextrin was purchased from Amazon.com and dissolved in water at 20% w/v. The concentration of the mature Tgase in the solution was confirmed by high-performance liquid chromatography (HPLC) and the sample was further diluted to 0.1 g/L of enzyme. Following dilution, the enzyme was subjected to the hydroxamate assay as described in Example 4 to determine specific activity. The specific activity of the commercial wild-type Tgase compared to Tgase variant A3 (SEQ ID NO: 4), further comprising an N-terminal methionine, is shown in FIG. 1.

### B. Thermal Stability

The thermal stability of Tgase variant A3 (SEQ ID NO: 4) compared to the commercial wild-type Tgase was evaluated by incubating the samples at 55° C. and testing the enzyme concentration and activity at various timepoints. Six timepoints were tested: 0, 30, 60, 120, 240, and 480 minutes.

The enzyme concentration was measured by HPLC. The samples were diluted 10-fold in 50 mM phosphate pH 7.4, 100 mM NaCl. The samples were tested by reverse phase using a Zorbax SB300-C8 column with 0.1% trifluoroacetic acid in water (MPA) and 0.08% trifluoroacetic acid in acetonitrile (MPB). Table 9 shows the gradient used during the HPLC run.

TABLE 9
HPLC gradient for determining Tgase concentration

| Time (min) | A | B | Flow Rate (mL/min) |
|---|---|---|---|
| 0 | 72.5 | 27.5 | 1 |
| 8 | 57.5 | 42.5 | 1 |
| 8.01 | 5 | 95 | 1 |
| 10 | 5 | 95 | 1 |
| 10.01 | 72.5 | 27.5 | 1 |
| 12 | 72.5 | 27.5 | 1 |

The activity was determined using the colorimetric activity assay described in Example 4A. The specific activity (U/mg) of the samples was calculated using the concentration of each sample as determined per Example 4D (results shown in Table 10). A greater portion of Tgase variant A3 (SEQ ID NO: 4) remained soluble during the heat treatment, indicating greater stability, the portion that remained in solution also maintained a higher specific activity (U/mg) at every timepoint tested, further indicating greater stability of Tgase variant A3 compared to wild-type Tgase.

TABLE 10
Specific activity of Tgase variant A3 (SEQ
ID NO: 4) compared to wild-type Tgase

Specific activity (U/mg)

| | Tgase variant A3 | |
|---|---|---|
| Timepoint (min) | (SEQ ID NO: 4) | Wild-type Tgase |
| 0 | 46.0 | 23.3 |
| 30 | 44.4 | 31.0 |
| 60 | 43.3 | 27.0 |
| 120 | 40.5 | 29.3 |
| 240 | 38.9 | 36.8 |
| 480 | 32.1 | 21.2 |

### C. Collagen-Modifying Activity

Substrate specificity for collagen of Tgase variant A3 (SEQ ID NO: 4) was tested by allowing the variant to react with either fluorescein-labeled gelatin (FITC-Gelatin, to test protein-protein cross-linking) or fluorescein-labeled cadaverine (FITC-cadaverine, to test protein-small molecule cross-linking) with a collagen layer deposited on the bottom of a 96-well microtiter plate. Tgase variant A3 (SEQ ID NO: 4) and wild-type Tgase were diluted to 4-800 ppm in 50 mM phosphate buffer, pH 7.4, supplemented with 100 mM NaCl in 96-well collagen coated microtiter plates. Either FITC-gelatin or FITC-cadaverine was added to 1 g/L (final volume 100 μL) and the plates were sealed and incubated at either 37° C. or 50° C. for 16 hours. The reaction supernatants were carefully removed and then 200 μL 50 mM phosphate, 100 mM NaCl, pH 7.4 buffer was added gently against the side of each well then carefully removed. This wash step was repeated. The plate-bound material was extracted by vigorously pipetting 100 μL dimethylsulfoxide into each well, then the extract was transferred into a black bottom 96-well plate. The fluorescence of the samples was measured at an excitation wavelength of 487 nm and an emission wavelength of 528 nm. The fluorescence signal is assumed to be proportionate to the concentration of fluorescent product extracted from the surface of the plate. For both FITC-gelatin and FITC-cadaverine, the fluorescence response per concentration enzyme was higher for wells treated with Tgase variant A3 (SEQ ID NO: 4) than for wells treated with wild-type Tgase (Table 11).

TABLE 11
Fluorescent values show collagen modifying activity

| | | | Fluorescence/ |
|---|---|---|---|
| | | Temperature | [Enzyme] |
| Substrate | Enzyme | (°C) | (RFU/ppm) |
| FITC-Gelatin | Tgase variant A3 | 37 | 8.4 |
| | (SEQ ID NO: 4) | 50 | 36.9 |
| | Wild-type Tgase | 37 | 4.6 |
| | | 50 | 6.8 |
| FITC- | Tgase variant A3 | 37 | 20.3 |
| Cadaverine | (SEQ ID NO: 4) | 50 | 182.2 |
| | Wild-type Tgase | 37 | 6.7 |
| | | 50 | 4.4 |

### Example 7. Testing Tgase Variants with Antimicrobial Chemicals for Aseptilase Activity

### A. Preparation of Challenge Substances

Capryl hydroxamic acid (CHA) was dissolved at approximately 3 wt % in a blend of 2 parts 1,3-propanediol and 1 part 1,2-hexanediol.

Coco Betaine was obtained as a 30 wt % solution in water.

The Tgase variants having the sequences disclosed herein as SEQ ID NOs: 3, 4, 5, and 6, respectively, were purified as described above and stored at a final concentration of 10 g/L, and then subjected to the Challenge Test.

### B. Challenge Test

For bactericidal testing, E. coli (ATCC 8739) was grown overnight (16-18 hours) in Mueller Hinton broth at 37° C. The following day, the cell density of the saturated cultures was calculated using OD₆₀₀, and approximately 5.0×10¹⁰ CFU bacteria were transferred to a sterile centrifuge tube, centrifuged at 4,000 rcf for 15 minutes to pellet the cells. Pellets were washed once using approximately 40 mL of sterile Butterfield's phosphate dilution buffer (BPDB), pelleted again, resuspended at a final concentration of approximately 1.0×10⁹ CFU/mL in sterile BPDB, and separated into 0.5 mL aliquots.

An aliquot of E. coli was challenged with 1.0% of the above CHA blend (300 ppm active), 1% of the above coco betaine solution (300 ppm active), 0.5% of the above Tgase solution (50 ppm active enzyme variant), or a combination thereof. Aliquots containing E. coli and challenge substance(s) were incubated at room temperature for one hour. Following incubation, samples were centrifuged at 21,000 rcf for 30 seconds to pellet cells and pellets were washed twice with sterile BPDB to remove all challenge substance(s) before finally resuspending at their original volume of 0.5 mL in BPDB for enumeration.

To enumerate surviving cell population, 100 μL of each sample was transferred in quadruplicate into 96-well plates and subjected to 10-fold serial dilutions with BPDB. Following serial dilution, an equal volume of Mueller Hinton broth containing resazurin to monitor cell viability was added to each well. Plates were sealed using clear film and allowed to incubate for 24 hours at 37° C. Following incubation, cell viability was assessed by monitoring conversion of resazurin to resorufin (OD₄₀₀), and most probable number (MPN) was calculated based on the number of dilutions required to dilute out the challenged population.

### Example 8. Aseptilase Activity of a Tgase Variant when Combined with Permeabilizers

Aseptilase activities of Tgase variant A3 (SEQ ID NO: 4) in combination with each of the three permeabilizer co-dosants, polymyxin B, chlorhexidine, and a 10000:4000:1 mixture by weight of sodium hexametaphosphate, phytic acid and chlorhexidine, were tested. A3 (SEQ ID NO: 4) was further improved by co-dosing with cell membrane permeabilizers. The experiments were performed as described in Example 5. The co-dosants were added into Tgase variant A3 (SEQ ID NO: 4) solutions at concentrations of 0.125 g/L, 0.05 g/L, and 350 mM, respectively. The results are shown in Table 12 with Tgase concentrations reported within the table at the best MIC.

TABLE 12
MIC values

| | Co-Dosant MIC in ppm | |
|---|---|---|
| | With Tgase variant | Without Tgase |
| Co-Dosant | (concentration of Tgase in ppm) | variant |
| Polymyxin B | 0.156 (12.5) | 0.625 |
| Chlorhexidine | 1 (200) | 2 |
| Hexametaphosphate, | 7.25^{a} (200) | >14.5^{a, b} |
| phytic acid, and | | |
| chlorhexidine | | |

^{a}The MIC value represents concentration of a mixture of 10000:4000:1 ratio by weight of hexametaphosphate, phytic acid, and chlorhexidine respectively.
^{b}Growth was observed at all codosants tested without Tgase variant A3 (SEQ ID NO: 4).

### Example 9. Boosting and/or Synergistic Effects of Aseptilase in Personal Care Product

Tgase variant A3 (SEQ ID NO: 4), referred to as aseptilase herein, was used in this example.

### A. Body Lotion

A vessel was filled with deionized water, placed under an overhead stirrer set to moderate mixing, and heated to 75-80° C. Glycerin was added to 2% and mixed until dissolved, followed by xanthan gum to 0.2%. At 75-80° C., cetearyl glucoside and cetearyl alcohol were added to 4% and 3%. Once both had melted fully, milk thistle seed oil was added and the batch was homogenized with a Silverson homogenizer with the standard emulsion screen until the batch had turned milky white. The batch was removed from heat and returned to propellor mixing with decreased speed to allow the emulsion to set up. At 40° C., pH was adjusted to pH 5.5-6.0 using citric acid or sodium hydroxide and split into several portions and allowed to finish cooling. Once cooled to room temperature, one portion of the body lotion was left unpreserved and the remaining portions were preserved with one or more substances selected from aseptilase, phenoxyethanol, caprylyl glycol, sodium benzoate, and EDTA. All samples were inoculated with E. coli (10⁶ CFU/mL) and C. albicans (10⁵ CFU/mL) and plated after 2 and 7 days. The results are reported as log reduction in CFU/mL in Tables 13A and 13B below.

TABLE 13A
Preservative efficacy against E. coli

| Condition | Day 2 | Day 7 |
|---|---|---|
| Unpreserved | −1.05 | −1.23 |
| 0.01% aseptilase | 1.71 | 3.45 |
| 0.005% aseptilase + 0.3% phenoxyethanol | 1.80 | 2.35 |
| 0.7% phenoxyethanol | 2.15 | 3.00 |
| 0.005% aseptilase + 0.35% caprylyl glycol | 4.00 | 4.00 |
| 0.7% caprylyl glycol | 5.00 | 5.00 |
| 0.005% aseptilase + | 3.06 | 5.36 |
| 0.5% sodium benzoate + 0.5% EDTA | | |
| 0.5% sodium benzoate + 0.5% EDTA | 0.83 | 2.94 |
| 0.005% aseptilase + 2% 2:1 Propanediol:Hexanediol | 1.80 | 2.60 |
| 0.005% aseptilase + 0.5% CHA solution | 2.07 | 2.69 |

TABLE 13B
Preservative efficacy against C. albicans

| Condition | Day 2 | Day 7 |
|---|---|---|
| Unpreserved | −1.05 | −1.08 |
| 0.01% aseptilase | −1.08 | −1.19 |
| 0.005% aseptilase + 0.3% phenoxyethanol | 0.25 | −0.03 |
| 0.7% phenoxyethanol | 0.68 | 1.73 |
| 0.005% aseptilase + 0.35% caprylyl glycol | 1.53 | 4.14 |
| 0.7% caprylyl glycol | 1.18 | 2.70 |
| 0.005% aseptilase + | 2.45 | 5.15 |
| 0.5% sodium benzoate + 0.5% EDTA | | |
| 0.5% sodium benzoate + 0.5% EDTA | 3.16 | 4.99 |
| 0.005% aseptilase + 2% 2:1 propanediol:hexanediol | −0.50 | −1.06 |
| 0.005% aseptilase + 0.5% CHA solution | 0.16 | 0.27 |

PCPC M-3 (or USP <51>) testing are performed in lotion samples (unpreserved and preserved) by a third-party microbiology lab.

### Example 10. Semipermenant Protein-Films for Hair and Textiles

### A. Color Retention:

The color retention on human hair was evaluated using Black Type 1 hair swatches purchased from Mayvenn (Mansfield, TX). The hair swatches each weighed approximately 2 g with a length of approximately 10 inches. The hair swatches were washed with 0.5 g of 20% SLES (Sodium Laureth Sulfate) and blow dried at 50° C. The hair swatches were then bleached twice with Kaleidocolors Clear Ice Powder Lightener mixed in a 1:2 ratio with 40 Volume Wella ColorCharm Cream Developer, wrapped in aluminum foil, and incubated at 25° C. for 45 minutes. The bleach was rinsed from the hair swatches and blow dried at 50° C. A L'Oreal Feria Cherry Crush hair coloring kit was applied to the hair swatches according to the instructions provided. The first color treated hair swatch (“control”) was rinsed and subsequently washed with the shampoo and conditioner provided by the hair coloring kit. Then the hair swatch was washed 10 times using 10% SLES and blow dried at 50° C. in between washes. The second color treated hair swatch was rinsed and subsequently washed with shampoo provided by the hair coloring kit. A total volume of 2 mL composed of 1000 ppm of the Tgase variant A3 (SEQ ID NO: 4) and 2% hydrolyzed keratin was placed on the hair swatch which was subsequently wrapped in foil and incubated at 50° C. for 30 minutes. The hair swatch was then rinsed followed by 10 washes with 10% SLES and blow dried at 50° C. in between washes.

After each wash and dry step, the rinse-off was collected and visually analyzed to assess color retention, shine, and frizz. The hair swatch treated with Tgase variant A3 (SEQ ID NO: 4) and hydrolyzed keratin retained color longer, felt smoother to touch, and had an added sheen that gave an overall healthier appearance over the control. Table 14A shows the absorbance values at 530 nm of the rinse-off collected following each washing step. Table 14B shows the visual observation of the red dye retained on the hair swatches. Data in Tables 14a and 14b demonstrate that color was retained longer on the hair swatch treated with Tgase variant A3 (SEQ ID NO: 4) than the control.

TABLE 14A
Absorbance values at 530 nm of the rinse-
off collected from washes of hair swatches

| | | Tgase variant A3 |
|---|---|---|
| | | (SEQ ID NO: 4)* and |
| Wash | | Hydrolyzed |
| No. | Control | Keratin** |
| 1 | 1.251 | 1.324 |
| 2 | 0.682 | 1.116 |
| 3 | 0.297 | 0.748 |
| 4 | 0.166 | 0.497 |
| 5 | 0.154 | 0.385 |
| 6 | 0.152 | 0.35 |
| 10 | 0.121 | 0.251 |

*1000 ppm of Tgase variant A3 (SEQ ID NO: 4)
**2% Hydrolyzed Keratin

TABLE 14B
Visual observation of color retained on hair after washes

| | | Tgase variant A3 |
|---|---|---|
| | | (SEQ ID NO: 4)* and |
| Wash | | Hydrolyzed |
| No. | Control | Keratin** |
| 1 | +++ | +++ |
| 2 | + | +++ |
| 3 | + | ++ |
| 4 | − | ++ |
| 5 | − | ++ |
| 6 | − | ++ |
| 10 | − | + |

+++: saturated red color on hair swatch
++: less saturated red color on hair swatch
+: very little red color on hair swatch
−: no detectable red color on hair swatch
*1000 ppm of Tgase variant A3 (SEQ ID NO: 4)
**2% Hydrolyzed Keratin

### B. Frizz Reduction and Style Retention

The frizz reduction on human hair was tested on Black Type 3 Curly hair swatches purchased from Mayvenn (Mansfield, TX) weighing approximately 2 g and having a length of approximately 10 inches. The hair swatches were washed with 0.5 g of 20% SLES. 2 mL of a treatment containing 1000 ppm Tgase variant A3 (SEQ ID NO: 4) in combination with each of the hydrolyzed proteins listed in Table 15A was applied to damp hair swatches. Control hair swatches were treated with water or each of the hydrolyzed proteins listed in Table 15. The hair swatches were dried at 50° C. and subsequently exposed to 85% relative humidity (RH) at 25° C. for 16 hours. Then, the hair swatches were soaked in water and dried at 50° C.

A noticeable difference in hair quality between the hair swatches treated with the combination of the Tgase variant A3 (SEQ ID NO: 4) and hydrolyzed proteins versus the controls was observed, and the data are shown in Tables 15B and 15C. The swatches treated with the Tgase variant A3 (SEQ ID NO: 4) in combination with a hydrolyzed protein were less frizzy, appeared hydrated, and exhibited more shine and curl definition than the control hair swatches after 16 hours at 85% RH and after the water soak.

TABLE 15A
Hydrolyzed proteins and dosages tested

| | | Percent |
|---|---|---|
| Substrate | | Hydrolyzed |
| No. | Hydrolyzed Proteins (INCI Name) | Protein (%) |
| 1 | Hydrolyzed Keratin | 0.4-1.15 |
| 2 | Wheat Amino Acids (and) Soy Amino | 0.065-1.65 |
| | Acids (and) Arginine | |
| | HCl (and) Serine (and) Threonine | |
| 3 | Hydrolyzed Wheat Protein | 0.1-1 |
| 4 | Hydrolyzed Soy Protein | 1 |
| 5 | Sodium Caseinate | 1 |
| 6 | Hydrolyzed Silk Protein | 0.04-1.4 |
| 7 | Potassium Cocoyl Hydrolyzed Collagen | 0.34-3.6 |

TABLE 15B
Treatments and observations after 16-hour at 85% RH at 25 °C

| | Frizz | Hydration | Increased |
|---|---|---|---|
| Treatment* | Reduction | Appearance | Shine |
| Tgase variant A3 (SEQ ID | + | + | + |
| NO: 4) + Substrate No. 1 | | | |
| Tgase variant A3 (SEQ ID | + | + | + |
| NO: 4) + Substrate No. 2 | | | |
| Tgase variant A3 (SEQ ID | + | + | + |
| NO: 4) + Substrate No. 3 | | | |
| Tgase variant A3 (SEQ ID | + | + | + |
| NO: 4) + Substrate No. 4 | | | |
| Tgase variant A3 (SEQ ID | + | + | + |
| NO: 4) + Substrate No. 5 | | | |
| Tgase variant A3 (SEQ ID | + | + | + |
| NO: 4) + Substrate No. 6 | | | |
| Tgase variant A3 (SEQ ID | + | + | + |
| NO: 4) + Substrate No. 7 | | | |
| Substrate No. 1 | − | − | − |
| Substrate No. 2 | − | − | − |
| Substrate No. 3 | − | − | − |
| Substrate No. 4 | − | − | − |
| Substrate No. 5 | − | − | − |
| Substrate No. 6 | − | − | − |
| Substrate No. 7 | − | − | − |
| Water | − | − | − |

*Tgase variant A3 (SEQ ID NO: 4) at 1000 ppm
+: presence
−: absence

TABLE 15C
Treatments and observations after water soak and drying at 50 °C

| | Frizz | Hydration | Increased |
|---|---|---|---|
| Treatment* | Reduction | Appearance | Shine |
| Tgase variant A3 (SEQ ID | + | + | + |
| NO: 4) + Substrate No. 1 | | | |
| Tgase variant A3 (SEQ ID | + | + | + |
| NO: 4) + Substrate No. 2 | | | |
| Tgase variant A3 (SEQ ID | + | + | + |
| NO: 4) + Substrate No. 3 | | | |
| Tgase variant A3 (SEQ ID | + | + | + |
| NO: 4) + Substrate No. 4 | | | |
| Tgase variant A3 (SEQ ID | + | + | + |
| NO: 4) + Substrate No. 5 | | | |
| Tgase variant A3 (SEQ ID | + | + | + |
| NO: 4) + Substrate No. 6 | | | |
| Tgase variant A3 (SEQ ID | + | + | + |
| NO: 4) + Substrate No. 7 | | | |
| Substrate No. 1 | − | − | − |
| Substrate No. 2 | − | − | − |
| Substrate No. 3 | − | − | − |
| Substrate No. 4 | − | − | − |
| Substrate No. 5 | − | − | − |
| Substrate No. 6 | − | − | − |
| Substrate No. 7 | − | − | − |
| Water | − | − | − |

*Tgase variant A3 (SEQ ID NO: 4) at 1000 ppm
+: presence
−: absence

### C. Hydrophobicity

Hydrophobicity of hair was evaluated using black Type 3 Curly hair swatches purchased from Mayvenn (Mansfield, TX) weighing approximately 2 g and having a length of approximately 10 inches. The hair swatches were washed with 0.5 g of 20% SLES. To damp hair swatches, 2 mL of a treatment was applied containing 100 ppm Tgase variant A3 (SEQ ID NO: 4) in combination with each of the hydrolyzed proteins listed in Table 15A. Control hair swatches were treated with water, each of the hydrolyzed proteins listed in Table 15a, and a leave-in conditioner formulated according to Table 16A. To prepare the leave-in conditioner, a vessel was with deionized water, heated to 75-80° C. with moderate mixing. Glycerin was added and mixed until dissolved. Phenoxyethanol and Ethylhexylglycerin were then added, which turned the solution hazy. In a separate beaker, Behentrimonium Methosulfate, Cetyl Alcohol, C13-15 Alkane, and Cetearyl Alcohol were pre-mixed and heated to 75-80° C. When the ingredients in both containers were fully melted and reached the set temperature, the pre-mix into the vessel at mixed at an increased mixing speed, which turned the solution to slightly translucent white. The solution was homogenized briefly with a Silverson homogenizer using a standard emulsion screen or immersion blender. After the solution turned milky white, which indicated that the emulsion had formed, the solution was returned to propellor mixing with a strong speed. The solution was then cooled to 50° C. with a slower mixing speed. The mixing speed at 50° C. was adjusted so that only the “top” of the mixture was moving, which allowed the emulsion and structurants to form and create thickening. The solution was side-swept with a spatula, if necessary, as the outside wall of the vessel would cool first. The faster the mixing below 50° C., the thinner the product would be. Homogenization was avoided as it would destroy the lamellar structure that was being formed. At 35° C., pH value of the solution was adjusted, if need, with citric acid pre-mixed in water.

The hair swatches were dried at 50° C. and subsequently placed in a beaker of water for 3 minutes. After 3 minutes, the position of the hair in the water was noted. The data are shown in Table 16B.

The hair swatches treated with Tgase variant A3 (SEQ ID NO: 4) floated near the surface of the water showing that a hydrophobic film had formed around the hair shaft with the ability to repel water. The control hair swatches sank to the bottom of the beaker showing that the hair was hydrophilic and absorbed the water from the beaker. The leave-in conditioner swatch behaved similarly to the Tgase variant A3 hair swatches.

TABLE 16A
Leave-In conditioner formula

| | % composition | |
|---|---|---|
| Ingredient | (w/w) | Purpose |
| Water | Q.S. to 100% | Carrier |
| Glycerin | 1% | Humectant |
| Phenoxyethanol, Ethylhexylglycerin | 1% | Preservative |
| Behentrimonium Methosulfate, Cetyl | 4% | Emulsifier |
| Alcohol | | |
| C13-15 Alkane | 4% | Emollient |
| Cetearyl Alcohol | 1% | Emollient |
| Citric Acid | Q.S. | pH Adjuster |

TABLE 16B
Treatments and observations of hair swatch positions in water

| | Position in |
|---|---|
| Treatment* | Water |
| Tgase variant A3 (SEQ ID NO: 4) + Substrate No. 1 | Floated |
| Tgase variant A3 (SEQ ID NO: 4) + Substrate No. 2 | Floated |
| Tgase variant A3 (SEQ ID NO: 4) + Substrate No. 3 | Floated |
| Tgase variant A3 (SEQ ID NO: 4) + Substrate No. 4 | Floated |
| Tgase variant A3 (SEQ ID NO: 4) + Substrate No. 5 | Floated |
| Tgase variant A3 (SEQ ID NO: 4) + Substrate No. 6 | Floated |
| Tgase variant A3 (SEQ ID NO: 4) + Substrate No. 7 | Floated |
| Substrate No. 1 | Sank |
| Substrate No. 2 | Sank |
| Substrate No. 3 | Sank |
| Substrate No. 4 | Sank |
| Substrate No. 5 | Sank |
| Substrate No. 6 | Sank |
| Substrate No. 7 | Sank |
| Water | Sank |
| Leave-In Conditioner | Floated |

*Tgase variant A3 (SEQ ID NO: 4) at 100 ppm

### D. Enhancing Color Retention and Strength of Wool

Color retention, fraying, and durability are evaluated on wool swatches (8 cm by 8 cm) obtained from Testfabrics, Inc. (West Pittston, PA). Each wool swatch is treated with 10-10,000 ppm of Tgase variant A3 (SEQ ID NO: 4) and optionally 0.5-4% of the hydrolyzed proteins described in Table 15A. The control wool swatch is left untreated. All wool swatches are washed with a commercially available laundry detergent. Wool swatches are visibly inspected for color retention, fraying, and durability after wash. Scanning electron microscopy is also performed on the wool swatches to evaluate the fraying on the surface of the wool fibers.

### Example 11. Increasing Structural Durability of Leather

### A. Metal-Free Hide Tanning

Hides that have been soaked, limed, de-limed, bated, and pickled are secured. The hides are treated with Tgase variant A3 (SEQ ID NO: 4) at a concentration range of 0.01-5% w/w relative to the weight of the hide and the temperature of the solution is adjusted to 4-50° C. Tanning is allowed to proceed for up to 24 hours and the tanning process is terminated by the enzyme from the hide. Leather properties are tested following standard methods such as ISO 3380:2016. Samples of treated leather are further tested by submerging them in water for 10 minutes at 70° C., 80° C., or 95° C. then measuring for shrinkage relative to un-tanned hide and conventional chromium tanned hide (wet-blue hide).

B. Metal-Free Hide Tanning with Crosslinker Extension

Amine substrates such as 1,4-butanediamine, 1,5-pentanediamine, 1,6-hexanediamine, 1,8-octanediamine, and diethylenetriamine are prepared in water at a concentration of 0.1-25% w/w relative to the weight of the hide. If necessary, an acid such as citric acid, acetic acid, or adipic acid is used to adjust the pH of the solution to 5.0-9.0. Hides are soaked in the pH-adjusted substrate solutions and Tgase variant A3 (SEQ ID NO: 4) is added at 0.01-5% w/w relative to the weight of the hide. The temperature of the solution is adjusted to 4-50° C. and tanning is allowed to proceed for up to 24 hours. Leather properties are tested following standard methods such as ISO 3380:2016. Samples of treated leather are tested by submerging them in water for 10 minutes at 70° C., 80° C., or 95° C. then measuring for shrinkage.

### C. Leather Repair

100 μL micellar casein in water (140 g/L) was applied to lacerations in leather with (Treatment A) or without (Treatment B) 0.05% Tgase variant A3 (SEQ ID NO: 4). The treated leather was heated at 37° C. for one hour and observed and then manipulated to determine the strength of the repair. As shown in Table 17, Treatments A and B both sealed the laceration, but the laceration with Treatment B readily failed during manipulation, while the laceration repair with treatment A (containing Tgase) was difficult to tear and remained sealed. Additional treatments are tested using Tgase variant A3 (SEQ ID NO: 4) combined with amine compounds such as 1,4-butanediamine, 1,5-pentanediamine, 1,6-hexanediamine, or 1,8-octanediamine at a concentration of 0.01% to 25% by weight.

TABLE 17
Leather laceration sealing

| | Treatment A | Treatment B |
|---|---|---|
| Laceration Sealed | +++ | ++ |
| Laceration Sealed after manipulation | +++ | − |

+++ the laceration is visibly sealed without defect.
++ the laceration is sealed but with some visible laceration remaining.
+ the laceration is sealed with obvious defects.
− the laceration is not sealed

### D. Leather Coating

Treatments A and B described in Table 17 were applied to the surface of leather samples. The samples were incubated for 30 minutes at 37° cand repeatedly bended to crack the surface, stirred in water for 2 minutes, and then dried for 90 minutes at 37° C. The samples were further subjected to bending and were again stirred in water for 2 more minutes, and then dried again for 90 minutes at 37° C. The surface of the treated leather samples was visually investigated for quality of the coating. While samples subjected to Treatment B showed significant flaking of the casein coating, Treatment A tightly sealed casein to the surface of the leather. The leather samples are also tested by submerging them in water for 10 minutes at 70° C., 80° C., or 95° C. then measuring for shrinkage.

SEQUENCE LISTING

SEQ ID NO: 1 Wild-type Streptomyces mobaraensis Tgase zymogen
(Pre-Pro-Tgase; Uniprot P81453)
MRIRRRALVFATMSAVLCTAGFMPSAGEAAADNGAGEETKSYAETYRLTADDVAN
INALNESAPAASSAGPSFRAPDSDDRVTPPAEPLDRMPDPYRPSYGRAETVVNNYIRKW
QQVYSHRDGRKQQMTEEQREWLSYGCVGVTWVNSGQYPTNRLAFASEDEDRFKNELK
NGRPRSGETRAEFEGRVAKESFDEEKGFQRAREVASVMNRALENAHDESAYLDNLKKE
LANGNDALRNEDARSPFYSALRNTPSFKERNGGNHDPSRMKAVIYSKHFWSGQDRSSS
ADKRKYGDPDAFRPAPGTGLVDMSRDRNIPRSPTSPGEGFVNFDYGWFGAQTEADADK
TVWTHGNHYHAPNGSLGAMHVYESKFRNWSEGYSDFDRGAYVITFIPKSWNTAPDKV
KQGWP
SEQ ID NO: 2 Mature wild-type Streptomyces mobaraensis Tgase
DSDDRVTPPAEPLDRMPDPYRPSYGRAETVVNNYIRKWQQVYSHRDGRKQQMTEEQR
EWLSYGCVGVTWVNSGQYPTNRLAFASFDEDRFKNELKNGRPRSGETRAEFEGRVAKE
SFDEEKGFQRAREVASVMNRALENAHDESAYLDNLKKELANGNDALRNEDARSPFYSA
LRNTPSFKERNGGNHDPSRMKAVIYSKHFWSGQDRSSSADKRKYGDPDAFRPAPGTGL
VDMSRDRNIPRSPTSPGEGFVNFDYGWFGAQTEADADKTVWTHGNHYHAPNGSLGAM
HVYESKFRNWSEGYSDFDRGAYVITFIPKSWNTAPDKVKQGWP
SEQ ID NO: 3 Stabilized Streptomyces mobaraensis Tgase variant
DPDDRVTPPAEPLDRMPDPYRPSYGRAETVVNNYIRKWQQVYSHRDGRKSQMTEEQRE
WLSYGCVGVTWVNSGAYPTNRLAFASEDEDRFHNELKNGRPRSGETRAEFEGRVAKES
FDEEKGFLRAREVASVMNRALENAHDESAYLDNLRRELANGNDALRNEDARSPFYSAL
RNTPSFKERNGGNHDPSRMKAVIYSKHFWSGQDRSSSADKRKYGDPDAFRPAPGTGLV
DMSRDRNIPRSPTSPGEGFVNFDYGWFGAQTEADADKTVWTHGNHYHAPNGSLGAMH
VYESKFRNWSEGYSDFDRGAYVITFIPKSWNTAPDKVKQGWP
SEQ ID NO: 4 Streptomyces mobaraensis Tgase variant with improved activity
DPDDRVTPPAEPLDRMPDPYRPSYGRAETVVNNYIRKWQQVYSHRDGRKSQMTEEQRE
WLSYGCVGVTWVNSGAYPTNRLAFASEDEDRFHNELKNGRPRSGETRAEFEGRVAKES
FDEEKGFLRAREVASVMNRALENAHDESAYLDNLRRELANGNDALRNEDARSPFYSAL
RNTPSFKERNGGNHDPSRMKAVIYSKHFWSGQDRSSSADKRKYGDPDAFRPAPGTGLV
DMSRDRNIPRSPTSPGEGFVNFDYGWFGAQTEADADKTVWTHGNHYHAPMASLGAMH
VYESKFRNWSEGYSDFDRGAYVITFIPKSWNTAPDKVKQGWP
SEQ ID NO: 5 Streptomyces mobaraensis Tgase variant with improved activity
DPDDRVTPPAEPLWRMPDPYRPSYGRAETVVNNYIRKWQQVYSHRDGRKSQMTEEQR
EWLSYGCVGVTWVNSGAYPTNRLAFASFDEDRFHNELKNGRPRSGETRAEFEGRVAKE
SFDEEKGFLRAREVASVMNRALENAHDESAYLDNLRRELANGNDALRNEDWRSPFYSA
LRNTPSFKERNGGNHDPSRMKAVIYSKHFWSGQDRSSSADKRKYGDPDAFRPAPGTGL
VDMSRDRNIPRSPTSPGEGFVNFDYGWFGAQTEADADKTVWTHGNHYHAPMASLGAM
HVYESKFRNWVEGYSDFDRGAYVITFIPKSWNTAPDKVKQGWP
SEQ ID NO: 6 Recombinant zymogen-form Streptomyces mobaraensis Tgase
(pro-Tgase) variant
MDNGAGEETKSYAETYRLTADDVANINALNESAPAASSAGPSFRAPMDPDDRVTPP
AEPLDRMPDPYRPSYGRAETVVNNYIRKWQQVYSHRDGRKQQMTEEQREWLSYGCVG
VTWVNSGQYPTNRLAFASFDEDRFKNELKNGRPRSGETRAEFEGRVAKESFDEEKGFQ
RAREVASVMNRALENAHDESAYLDNLKKELANGNDALRNEDARSPFYSALRNTPSFKE
RNGGNHDPSRMKAVIYSKHFWSGQDRSSSADKRKYGDPDAFRPAPGTGLVDMSRDRNI
PRSPTSPGEGFVNFDYGWFGAQTEADADKTVWTHGNHYHAPNGSLGAMHVYESKERN
WSEGYSDFDRGAYVITFIPKSWNTAPDKVKQGWP
SEQ ID NO: 7 Pro-sequence from recombinant zymogen-form Streptomyces
mobaraensis Tgase (pro-Tgase) variant
MDNGAGEETKSYAETYRLTADDVANINALNESAPAASSAGPSFRAP

1. A transglutaminase variant comprising the sequence of SEQ ID NO: 2 further comprising at least one or more amino acid substitutions comprising one or more of the following:
substitution of S2 with P;
substitution of Q39 with N;
substitution of Q40 with E, F, H, K, L, or N;
substitution of K49 with H or R;
substitution of Q50 with A, H, N, S or T;
substitution of Q51 with A, C, D, E, G, I, K, P, or R;
substitution of Q56 with A, E, or M;
substitution of Q74 with A, D, E, F, G, H, I, L, M, N, R, S, T, V, or Y;
substitution of K91 with A, C, E, H, M, N, or Y;
substitution of K95 with I or R;
substitution of Q124 with A, E, I, L, M, or N;
substitution of K151 with R or Y;
substitution of K152 with R; or
substitution of Q328 with F, H, L, M, or P;
and, optionally, the one or more amino acid substitutions further comprising one or more of the following:
substitution of S2 with C, D, E, F, H, I, K, L, M, N, Q, R, T, V, W, or Y;
substitution of A10 with C, Q, or T;
substitution of D14 with H, L, M, N, W, or Y;
substitution of R15 with A, E, or T;
substitution of D18 with E or T;
substitution of G47 with H;
substitution of R48 with M;
substitution of K49 with E or T;
substitution of Q74 with C;
substitution of K95 with E;
substitution of S131 with E or I;
substitution of V132 with L;
substitution of N134 with S or T;
substitution of A136 with C or S;
substitution of L137 with K or V;
substitution of Y146 with F;
substitution of L147 with E or M;
substitution of E164 with F;
substitution of A166 with W;
substitution of P169 with E;
substitution of F170 with I, L, or V;
substitution of S199 with A or G;
substitution of N282 with D, E, K, M, Q, or R;
substitution of G283 with A;
substitution of S284 with A, D, E, or P;
substitution of H289 with E, I, L, Q, T, or V; and
substitution of S299 with A, E, K, or V.

2.-9. (canceled)

10. The transglutaminase variant of claim 1, comprising
S2P and Q50H;
S2P and Q74V;
S2P and Q50A;
S2P and Q51P;
S2P, Q39N, and Q51E;
S2P, Q56M, and Q74S;
S2P, K49T, and Q74M;
S2P, K49T, and Q74F;
S2P, Q511, and K951;
S2P, Q124E, K151R, and K152R;
S2P, Q124I, K151R, and K152R;
S2P, Q124L, K151R, and K152R;
S2P, Q124N, K151R, and K152R;
S2P, Q50S, Q51E, and Q56E;
S2P, Q51P, Q56M, Q74A, and K91E;
S2P, Q50S, Q74A, K91H, Q124L, K151R, and K152R;
S2P, Q50S, Q74A, K91H, Q124N, K151R, and K152R;
S2P, K49H, Q50A, Q74C, Q124L, K151R, and K152R;
S2P, K49H, Q50A, Q74C, Q124N, K151R, and K152R;
S2P, Q51A, Q74T, K951, Q124L, K151R, and K152R;
S2P, Q50T, Q51A, Q74M, K95E, Q124L, K151R, and K152R;
S2P, Q50S, Q74A, K91H, Q124L, K151R, K152R, and N282R;
S2P, Q50S, Q74A, K91H, Q124L, K151R, K152R, N282E, and S284A;
S2P, Q50S, Q74A, K91H, Q124L, K151R, K152R, N282M, and G283A;
S2P, Q50S, Q74A, K91H, Q124L, K151R, K152R, N282Q, S284A, and H2891;
S2P, Q50S, Q74A, K91H, Q124L, K151R, K152R, N282D, S284A, H2891, and S299E;
S2P, D14W, Q50S, Q74A, K91H, Q124L, K151R, K152R, A166W N282M, G283A, and S299V;
S2P, Q50S, Q74A, K91H, Q124L, K151R, K152R, N282M, G283A, and S299V;
S2P, D14W, Q50S, Q74A, K91H, Q124L, K151R, K152R, N282M, G283A, and S299A;
S2P, Q50S, Q74A, K91H, K95E, Q124L, K151R, K152R, N282M, G283A, and S299V;
S2P, A10T, Q50S, Q74A, K91H, Q124L, S1311, K151R, K152R, N282M, G283A, and S299V;
S2P, D14W, Q50S, Q74A, K91H, Q124L, S1311, K151R, K152R, N282M, G283A, and S299V;
S2P, R15T, Q50S, Q74A, K91H, Q124L, K151R, K152R, N282M, G283A, and H2891;
S2P, Q50S, Q51E, Q74A, K91H, Q124L, K151R, K152R, F1701, N282M, G283A, H2891, and S299V;
S2P, Q50S, Q51E, Q74A, K91H, Q124L, Y146F, K151R, K152R, N282M, G283A, H2891, and S299V;
S2P, Q50S, Q74A, K91H, K95E, Q124L, K151R, K152R, N282M, and G283A;
S2P, D18T, Q50S, Q74A, K91H, Q124L, S131R, V132L, K151R, K152R, N282M, and G283A
S2P, D14N, Q50S, Q74A, K91H, Q124L, K151R, K152R, N282M, and G283A; and
S2P, Q50S, Q51A, Q74A, K91H, Q124L, K151R, K152R, N282M, G283A, and H2891.

11. (canceled)

12. The transglutaminase variant of claim 1, wherein the variant comprises the sequence of any of SEQ ID NOS: 3-5.

13. The transglutaminase variant of claim 1, wherein the variant comprises the sequence of SEQ ID NO: 4.

14.-22. (canceled)

23. A composition comprising the transglutaminase variant of claim 1.

24. (canceled)

25. The composition of claim 23, further comprising a peptide, protein, or hydrolyzed protein at a concentration of about 0.04% to about 10%

26. The composition of claim 23, further comprising a diamine molecule, wherein, optionally, the diamine molecule is one or more of 1,4-butanediamine, 1,5-pentanediamine, 1,6-hexanediamine, 1,8-octanediamine, and diethylenetriamine.

27. A method of preserving or increasing the shelf life of a product comprising incorporating the transglutaminase variant of claim 1.

28. A method of forming a semipermanent protein-film on an object, comprising applying to the object the composition of claim 23.

29. The method of claim 28, wherein the object is hair, skin, textile, or leather.

30. The method of claim 28, wherein the composition comprises about 10 to about 10,000 ppm of the transglutaminase variant.

31. A method of retaining color and/or reducing color transfer on hair or skin, comprising applying to the hair or the skin a composition comprising about 10 to about 10,000 ppm of the transglutaminase variant of claim 1.

32. A method of reducing frizz and/or retaining style in hair, comprising applying to the hair a composition comprising about 10 to about 10,000 ppm of the transglutaminase variant of claim 1.

33. A method of improving hydrophobicity of hair, comprising applying to the hair a composition comprising about 10 to about 10,000 ppm of the transglutaminase variant of claim 1.

34. A method of retaining color and/or reducing color transfer on textile or leather, comprising applying to the textile or the leather a composition comprising about 10 to about 10,000 ppm of the transglutaminase variant of claim 1.

35. A method of strengthening and/or reducing fraying or peeling of textile, comprising applying to the textile a composition comprising about 10 to about 10,000 ppm of the transglutaminase variant of claim 1.

36. A method of improving water resistance and/or wrinkle resistance of textile, comprising applying to the textile a composition comprising about 10 to about 10,000 ppm of the transglutaminase variant of claim 1.

37. (canceled)

38. A method of tanning hide, comprising applying to the hide the transglutaminase variant of claim 1 at about 0.001% to about 5% w/w relative to the weight of the hide.

39. The method of claim 38, further comprising applying to the hide a diamine molecule at about 0.1% to about 25% w/w relative to the weight of the hide, wherein, optionally, the diamine molecule is one or more of 1,4-butanediamine, 1,5-pentanediamine, 1,6-hexanediamine, 1,8-octanediamine, and diethylenetriamine.

40. A method of repairing, coating, and/or improving water resistance of leather, comprising applying to the leather a composition comprising about 10 to about 10,000 ppm of the transglutaminase variant of claim 1.

41. (canceled)


SEQUENCE LISTING (7 sequences)

SEQ ID NO: 1 - protein, 407 aa, Streptomyces mobaraensis [sequence omitted]

SEQ ID NO: 2 - protein, 331 aa, Streptomyces mobaraensis
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 3 - protein, 331 aa, Synthetic construct
DPDDRVTPPAEPLDRMPDPYRPSYGRAETVVNNYIRKWQQVYSHRDGRKSQMTEEQREWLSYGCVGVTWVNSGAYPTNRLAFASFDEDRFHNELKNGRPRSGETRAEFEGRVAKESFDEEKGFLRAREVASVMNRALENAHDESAYLDNLRRELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 4 - protein, 331 aa, synthetic construct
DPDDRVTPPAEPLDRMPDPYRPSYGRAETVVNNYIRKWQQVYSHRDGRKSQMTEEQREWLSYGCVGVTWVNSGAYPTNRLAFASFDEDRFHNELKNGRPRSGETRAEFEGRVAKESFDEEKGFLRAREVASVMNRALENAHDESAYLDNLRRELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPMASLGAMHVYESKFRNWSEGYSDFDRGAYVITFIPKSWNTAPDKVKQGWP

SEQ ID NO: 5 - protein, 331 aa, synthetic construct
DPDDRVTPPAEPLWRMPDPYRPSYGRAETVVNNYIRKWQQVYSHRDGRKSQMTEEQREWLSYGCVGVTWVNSGAYPTNRLAFASFDEDRFHNELKNGRPRSGETRAEFEGRVAKESFDEEKGFLRAREVASVMNRALENAHDESAYLDNLRRELANGNDALRNEDWRSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPMASLGAMHVYESKFRNWVEGYSDFDRGAYVITFIPKSWNTAPDKVKQGWP

SEQ ID NO: 6 - protein, 378 aa, synthetic construct
MDNGAGEETKSYAETYRLTADDVANINALNESAPAASSAGPSFRAPMDPDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 7 - protein, 46 aa, synthetic construct
MDNGAGEETKSYAETYRLTADDVANINALNESAPAASSAGPSFRAP